(19) United States
(12) Patent Application Publication
(10) Pub. No.: US 2004/0203134 A1
(43) Pub. Date: Oct. 14, 2004

(54) Complex technologies using enzymatic protein hydrolysate

(76) Inventors: Alexander V. Pyntikov, Ashburn, VA (US); Salvatore P. Salerno, Herndon, VA (US)

Correspondence Address: HELLER EHRMAN WHITE & MCAULIFFE LLP, 1666 K STREET,NW, SUITE 300, WASHINGTON, DC, 20006, US

(21) Appl. No.: 10/358,136

(22) Filed: Feb. 5, 2003

Related U.S. Application Data
(60) Provisional application No. 60/354,270, filed on Feb. 6, 2002.

(51) Int. Cl. C12N 1/20; C12N 1/18

(52) U.S. Cl. 435/252.1; 435/254.1

(57) ABSTRACT

The present invention comprises various uses of enzymatic protein hydrolysate (“EPH,”). The EPH production process involves the profound enzymatic protein hydrolysis of marine animal biomass, whereby cold-water fish wastes are reacted with viscera containing aggressive enzymes that are effective in even mild (slightly alkaline) medium of water. The EPH may be dried to a powder state that is made of approximately 70%-90% free amino acids, 10%-20% highly molecular peptides and 3%-5% vitamins, minerals and oils. The biological value of the EPH stems from the special mix of nutrients and the method by which they are obtained. EPH is particularly suitable as an enhancer for the acceleration of known biological and industrial processes, and most particularly, processes depending upon bacterial or cellular action. The low cost of this highly nutritious product makes it possible to utilize mixtures of amino acids and other nutrients in bio-industrial processes to make them cost-competitive with competing processes.

### RELATED U.S. APPLICATION DATA

This application claims the benefit of provisional application Ser. No. 60/354,270 entitled Proteol[ytic] Fermenter And Complex Technologies Using The Proteol[ytic] Fermenter, filed Feb. 6, 2002.

### BACKGROUND OF THE INVENTION

1. Field of the Invention

The present invention relates to the use of an enzymatic protein hydrolysate as a catalyst in specified industrial applications involving cellular and bacterial growth, and more particularly, to the industrial uses of enzymatic hydrolysate of marine animal biomass.

2. Background

The elementary nutrients for cellular growth of life on Earth are amino acids. Every chemical structure in all living organisms (DNA, RNA, proteins, fats, carbohydrates, hormones, vitamins, and the amino acids themselves) are synthesized and broken down by means of enzymes that, in turn, are proteins, constructed from amino acids. For representatives of all the five kingdoms of living organisms, life on Earth essentially is the circulation of amino acids. For example, representatives of the kingdom of plants continuously make contributions of mass doses of amino acids to global amino acid quantities (by way of photosynthesis).

Food is digested in the human body into amino acids, simple sugars and fatty acids. Amino acids are not only food for human beings, they are also a base for the rapid accumulation of biomass of individual species of organisms in any of the five kingdoms of the terrestrial life. Humans can help protect their species against vagaries and catastrophes within any of the kingdoms of living things on Earth by creating a steadily renewable supply of amino acids. Humans can use this resource in many beneficial ways, such as for the production of meat, or natural gas, or alcohol, or for the purification of water or air, or for combating the greenhouse effect on Earth.

Methods of hydrolyzing proteins are well known in the art. It is also well known in the art to hydrolyze fish in an alkaline environment so to produce animal feed and other food products. However there are a number of fundamental problems in the hydrolysis of fish proteins using organic, fish based enzymes. Among those problems have been the inability to obtain a consistent mix of nutrient products on a large-scale basis, shortcomings in the art regarding concentrating, drying, separating, and purifying those products and high relative costs of production.

WIPO application number WO 01/28353 by Bjarnason, J. et. al., published Apr. 26, 2001, discloses a method for enzymatically obtaining protein hydrolysates for human consumption, animal feed, and cosmetics. Bjarnason et. al. do not disclose the method or products of the present invention.

U.S. Pat. No. 3,856,627 by Nagasawa et. al., patented Dec. 24, 1974, describes a culture media for bacteria containing fish protein hydrolysate. Nagasawa et. al., do not disclose the products or the uses of the present invention.

U.S. Pat. No. 5,821,111 by Grady, J. et. al., patented Oct. 13, 1998, describes a method for converting waste biomass to useful products. Grady et. al., do not disclose the products or the uses of the present invention.

U.S. Pat. No. 5,490,634 by Jain, M. et. al., patented Feb. 13, 1996, describes a biological method for coal comminution. Jain et. al., do not disclose the products or the uses of the present invention.

U.S. Pat. No. 5,480,865 by Kingham, D. et. al., patented Jan. 2, 1996, describes a nutritional composition for management of protein intake. Kingham et. al., do not disclose the products or the uses of the present invention.

U.S. Pat. No. 5,464,539 by Ueno, Y. et. al., patented Nov. 7, 1995, describes a process for the production of hydrogen by microorganisms. Ueno et. al., do not disclose the products or the uses of the present invention.

U.S. Pat. No. 5,094,668 by Kern, E. et. al., patented Mar. 10, 1992, describes a method of enzymatic coal desulfurization. Kern et. al., do not disclose the products or the uses of the present invention.

U.S. Pat. No. 4,960,699 by Wood, W. et. al., patented Oct. 2, 1990, describes a method for enzymatic depolymerization of coal. Wood et. al., do not disclose the products or the uses of the present invention.

U.S. Pat. No. 4,914,024 by Strandberg, G. et. al., patented Apr. 3, 1990, describes a method for microbial solubilization of coal. Strandberg et. al., do not disclose the products or the uses of the present invention.

U.S. Pat. No. 4,882,274 by Pyne, Jr., J. et. al., patented Nov. 21, 1989, describes a method for solubilization of low-rank coal using a cell-free enzymatic system. Pyne, Jr. et. al., do not disclose the products or the uses of the present invention.

U.S. Pat. No. 4,659,670 by Stevens, Jr., S. et. al., patented Apr. 21, 1987, describes a method for biological desulfurization of coal. Stevens, Jr., S. et. al., do not disclose the products or the uses of the present invention.

U.S. Pat. No. 4,540,666 by Nukina, Y. et. al., patented Sep. 10, 1985, describes a method for methane fermentation. Nukina et. aL, do not disclose the products or the uses of the present invention.

U.S. Pat. No. 4,317,843 by MacLennan, D. et. al., patented Mar. 2, 1982, describes a method for microbiological production of protein. MacLennan et. al., do not disclose the products or the uses of the present invention.

U.S. Pat. No. 4,294,856 by Kinumaki, T. et. al., patented Oct. 2, 1990, describes a method for manufacturing artificial milk replacer for raising infant pigs and other infant animals. Kinumaki et. al., do not disclose the products or the uses of the present invention.

U.S. Pat. No. 4,206,288 by Detz, C. et. al., patented Jun. 3, 1980, describes a method for microbial desulfurization of coal. Detz et. al., do not disclose the products or the uses of the present invention.

U.S. Pat. No. 4,041,182 by Erickson, L. et. al., patented Aug. 9, 1977, describes a method for manufacturing bio-protein feed. Erickson et. al., do not disclose the products or the uses of the present invention.

U.S. Pat. No. 3,970,520 by Feldman, J. et. al., patented Jul. 20, 1976, describes a method for preparing nutritionally improved foodstuffs. Feldman et. al., do not disclose the products or the uses of the present invention.

U.S. Pat. No. 5,905,033 by Morita, M. et. al., patented May 18, 1999, describes a method for obtaining a microbial culture medium from the entrails of fish, shellfish, or cephalopods and culturing microorganisms using same. Morita et. al., do not disclose the products or the uses of the present invention.

U.S. Pat. No. 5,536,645 by Jay, C., patented Jul. 16, 1996, describes a nutritive medium for the culture of microorganism. Jay does not disclose the products or the uses of the present invention.

U.S. Pat. No. 3,989,594 by MacLennan, D. et. al., patented Nov. 2, 1976, describes a method for microbiological production of protein. MacLennan et. al., do not disclose the products or the uses of the present invention.

U.S. Pat. No. 6,294,351 by Lin, M., et. al., patented Sep. 25, 2001, describes a method for biochemical transformation of solid carbonaceous material. Lin et. al., do not disclose the products or the uses of the present invention.

U.S. Pat. No. 6,130,076 by Ingram, L., patented Oct. 10, 2000, describes a method for producing ethanol using a soy hydrolysate-based medium or a yeast autolysate-based medium. Ingram does not disclose the products or the uses of the present invention.

U.S. Pat. No. 6,084,139 by Van der Giessen, A., et. al., patented Jul. 4, 2000, describes a method for processing waste of biomass material. Van der Giessen et. al., do not disclose the products or the uses of the present invention.

U.S. Pat. No. 5,981,266 by Srivastava, K., et. al., patented Nov. 9, 1999, describes a microbial process for mitigating sulfur compounds from natural gas. Srivastava et. al., do not disclose the products or the uses of the present invention.

U.S. Pat. No. 5,885,825 by Lin, M., et. al., patented Mar. 23, 1999, describes a method for biochemical transformation of coals. Lin et al., do not disclose the products or the uses of the present invention.

Related Definitions

GROWTH PHASE: One of three dominant phases of microorganism growth. These three phases include: 1. microorganism reproduction; 2. sustained growth; and 3. declining growth. The growth phase may include any period of time in which the microorganisms are acclimating to a new food source.

PROTEINS: Proteins are high molecular weight organic compounds with molecular weights ranging from 5000 to many millions of daltons. Proteins are polymers (or copolymers), the monomers of which are amino acids connected to each other by peptide bonds. Peptide chains of smaller sizes (less than 5000 daltons) form peptide and polypeptide molecules. There are various principles for the classification of proteins based on composition, properties, where they are found in nature, and the functions they perform. With respect to composition, proteins are classified as simple or complex (proteins and proteids, or conjugated proteins). The composition of proteins includes, in addition to amino acids, components such as carbohydrates (glycoproteins), nucleic acids (nucleoproteins), fats (lipoproteins), phosphoric acid (phosphoproteins), metals (metalloproteins) or other compounds. One of the criteria used for classification is the solubility of the proteins in various solvents. Albumin and globulin, which dissolve in water and in weak aqueous solutions of electrolytes, are considered soluble proteins, although their stability in solution varies. At the opposite extreme are collagen and keratin—proteins which are insoluble in water and other solvents. In interaction with water, many of the insoluble proteins swell and form a gel (gelatinize). An additional criterion for classification is behavior in an electrical field, depending upon the total electrical charge of their molecules, which are polyions. The electrophoretic mobility of a protein at a specific pH level depends upon its amino acid composition and its isoelectric point (pI—the pH value of the medium at which the protein is electrically neutral). Basic, neutral and acidic proteins are distinguished according to their electrochemical properties. According to biological functions, proteins are classified as biologically active (enzymes, hormones), structural, regulatory, contractile, reserve, ovalbumin and casein type, transport (blood serum proteins, hemoglobin, myoglobin), protective (antibodies, blood coagulating proteins) and toxins. The whole spectrum of proteins is provided by the particular features of their structures. The protein part of the protein molecule is a polymer chain of amino acids connected by peptide bonds. Amino acids and peptides are formed in the hydrolysis of proteins.

AMINO ACIDS: Amino acids are optically active organic compounds containing an amino group and a carboxyl group at the carbon atom. In the condensation of two amino acids, the amino group of one of them enters into a linkage with the carboxyl group of the other. The linear sequence of amino acids in the polypeptide chain is considered the primary structure of the protein. Proteins are long-chain molecules that are intricately folded into 3-dimensional structures. The chain is composed of strings of small and fairly simple molecules called amino acids. The instructions for making proteins are carried by the DNA. In the 1960s the code for translating DNA sequences into protein structures was discovered, following intensive work since Watson and Crick's discovery of the double helical structure of the DNA molecule in 1953.The compositions of proteins include the 26 amino acids which are most common in nature in the stereo-isomeric form of L-isomers, and more than 10 rare and very rare amino acids. Out of the whole list, there are 8 amino acids that are essential; i.e., they cannot be synthesized in the bodies of animals and have to be obtained exclusively from their diet. Two amino acids are provisionally essential. The other amino acids can be synthesized in animal and plant organisms

PEPTIDES: Proteins are synthesized as a result of the formation of secondary amide bonds between carboxyl groups and amino groups of adjacent amino acids. Such bonds are called peptide bonds, and the structures which emerge as a result of the formation of the peptide bonds between amino acid radicals are called peptides. A peptide that contains two amino acid radicals is called a dipeptide; a peptide that contains three radicals is called a tripeptide, etc. The covalent peptide bonds and disulfide bonds with an energy greater than 35 kcal/mol are the most important bonds in the polypeptide chain. The peptide bonds [(C—N)-group] are partly double bonds and preclude the free rotation of the atoms. Since there is one peptide bond for every 3 bonds in the peptide chain, the free rotation is possible only around the other two bonds. As a result, the peptide chain is spontaneously twisted into a helix. The form of the spiral determines the nature of the secondary structure of the protein. Hydrogen bonds with an energy of 5 kcal/mol play a large role in the formation of the secondary structure. The method for the folding of the structure into its tertiary form is determined by weak interactions within the protein molecule: Coulomb (electrostatic) and Van der Waals. These bonds are very unstable under heating and, like hydrogen bonds, have a pronounced thermolabile nature. Nevertheless, all the other levels of structural organization and, consequently, the method of self-assembly of the molecules are programmed, as it were, in the amino acid sequences of the proteins. The accessibility of the peptide bonds to enzymes is of importance for the process of enzymatic hydrolysis. However, these bonds are normally masked inside a globule and are attached by hydrogen bonds to the neutral polar groups or form ion pairs. The denaturation of proteins results in unmasking of the susceptible bonds and opens them up to enzyme attack.

There are adequate industrial raw material sources of protein in all the industrially developed countries of the world. Protein sources may include wastes from the meat processing, fish processing and milk processing industries, vegetable proteins, as well as biomass from artificially cultivated microorganisms, fungi or one-celled algae. The criterion for the selection of proteins for hydrolysis is the nutritional value and completeness of the proteins in question. The nutritional value of proteins is determined at present by the calculation (scoring) method on the basis of the concentrations of essential amino acids. Essential amino acids pass into the human body and the bodies of animals with their diet, while bacterial and cell cultures receive them from the nutrient medium. The completeness of the proteins for nutritional purposes is determined by comparison to a protein that has been adopted as a standard. The calculation of the score made of the quantitative determination of essential amino acids in the protein and the determination of the ratio of the concentration of each to the corresponding concentration in a standard specimen or model mixture. The value of the minimum ratio, i.e., the ratio for the limiting amino acid, is accepted as the score. Proteins from hen's eggs, mother's milk and goose eggs have been adopted as standard proteins. The process for the calculation of corrected amino acid scoring of protein compositions—i.e., the calculation of the score with correction for the susceptibility to breaking down (hydrolysis) of the protein-has been accepted since 1993.

Muscle tissue is a source of proteins which are the most balanced in regard to their amino acid composition, as well as mineral elements, vitamins and growth factors, including substances of a nature which is not yet known. Most biotechnologists have believed and continue to believe that meat cannot be replaced as a source of protein for nutrient media. At the same time, meat is one of the main food products. Therefore, meat is used extremely rarely as a source of raw material in the industrial production of hydrolysates, and it is used as efficiently as possible.

HYDROLYSIS: The hydrolysis of proteins is the splitting of the molecule with the destruction of its primary structure (the polypeptide chain). The hydrolysis of the peptide chain is the reverse of the process of formation of the peptide chain; it occurs as a result of the effects of physical factors (temperature, pressure), chemical reagents (acids, bases) or proteolytic enzymes. As a result of the hydrolysis of a dipeptide, two amino acid molecules are formed, and one molecule of water is absorbed: R—CHNH2-CO—NH—R—COOH+H—OH>R—CHNH2-COOH+NH2-R—COOH. The products of the full hydrolysis of proteins are amino acids; the products of partial hydrolysis are amino acids and peptides. Typically it is chemical or enzymatic methods that are used for hydrolysis. The chemical methods are used for full (total) hydrolysis of proteins and for partial hydrolysis and are also used in certain cases for the spot (selective) breaking of peptide bonds. The methods of full hydrolysis are used, as a rule, for purposes of analysis. Acid hydrolysis is used often under industrial conditions; alkali hydrolysis is more rarely used.

ACID HYDROLYSIS is conducted primarily with the use of mineral acids at high temperatures (acid-thermal hydrolysis). With the effect of high concentrations of the acids on the protein under heating for a sufficiently long period of time, the protein can be broken down completely into amino acids. Acid hydrolysis is normally performed with sulfuric or hydrochloric acid. An attractive aspect of acid hydrolysis is the possibility of obtaining deep hydrolysates in short periods of time. Another positive factor is the establishment of bactericide conditions in the course of the process, which prevents bacterial growth and makes it possible to store the hydrolysate for a long time without neutralization. However, acid hydrolysis also has its negative aspects. Since acid hydrolysis is not specific to proteins, in the acid-thermal treatment of a complex, mixed raw material of the kind normally used in industry, the breaking down of other biological polymers is also going on at the same time: nucleic acids and polysacharrides. As a result of the hydrolysis of such raw material, the hydrolysis products turn out to be not just amino acids and peptides but carbohydrates (monoses and reducing disacharrides). In acid hydrolysis, melanoids are formed-dark-colored, high molecular weight compounds with a tendency toward aggregation, which have poor solubility in water. The melanoids are cell toxins; therefore, their formation in the hydrolysate sharply reduces its quality. The neutralization of acids in the hydrolysate involves the formation of high concentrations of salts. The higher the concentration of the acid used for hydrolysis, the more salts are formed as a result of neutralization. The increased anion concentration is also a salient factor in the toxicity of the hydrolysate.

ALKALI HYDROLYSIS: In the alkali hydrolysis of proteins, most amino acids are broken down and more importantly, the method for alkali hydrolysis is softer and gentler than acid hydrolysis. As a result, alkali hydrolysis is best used in the industrial production of hydrolysates. Of the known means to hydrolyze proteins, enzymatic hydrolysis in a slightly alkaline environment at a moderate temperature, is preferred since these conditions do not significantly destroy essential amino acids. However, in the hydrolysis of animal biomass, most animal-produced enzymes, or ferments, are not robust enough to hydrolyze animal proteins under such mild conditions. Hydrolysis occurs in pH ranges that correspond to the peaks in the activity of the enzymes: more often in a neutral, slightly alkaline medium. The optimum temperature is 35-50° C. This threshold can be raised to 60° C. for certain enzymes, but should not be higher than 80° C. Despite its relative advantages, enzymatic hydrolysis of proteins—using natural ferments—has typically resulted in products whose constituents cannot be accurately predicted on a consistent basis. Further difficulties in industrial scale enzymatic hydrolysis of animal proteins using natural ferments stems from the fact that cost-effective industrial dryer and micro-separation technologies have not been utilized for the production of contaminant-free high-grade amino acids in this environment.

### SUMMARY OF THE INVENTION

The present invention comprises various uses of enzymatic protein hydrolysate (“EPH,” hereinafter). EPH is a mixture of peptides, amino acids, vitamins, minerals, and specified elements. The EPH production process involves the enzymatic protein hydrolysis of marine animal biomass, whereby cold-water fish wastes are reacted with viscera containing aggressive enzymes that are effective in even mild (slightly alkaline) medium of water. Not only are the fish abundant, they are uniformly carnivorous. In particular, cold-water fish have natural enzymes enabling them to digest proteins under conditions of relatively cold temperature ranges.

The enzymatic protein hydrolysate (EPH), envisioned by the current invention, is produced by profound enzymatic protein hydrolysis of cold water animals. The EPH may be dried to a powder state. Dry EPH is made of amino acids, short peptides and macro and microelements in the approximate range of 70%-90% free amino acids, 10%-20% highly molecular peptides and 3%-5% vitamins, minerals and oils. The biological value of the EPH stems from the special mix of nutrients and the method by which they are obtained. EPH is particularly suitable as an enhancer for the acceleration of known biological and industrial processes, and most particularly, processes depending upon bacterial or cellular action.

It is an object of this invention to produce an enzymatic protein hydrolysate derived from cold water fish that can be used as a nutritious medium for cellular and bacterial growth in industrial processes.

It is a further object of this invention to mass produce a nutritious amino acid, vitamin, and mineral mix derived from cold-water marine animals, and utilize said mixture in a variety of nutritional and pharmaceutical applications.

It is a further object of this invention to mass produce a nutritious amino acid, vitamin, and mineral mix derived from cold-water marine animals, and utilize said mixture as a means to enhance the biological production, extraction and fermentation of natural gas and other fuels, including coal bed methane.

It is a further object of this invention to mass produce a nutritious amino acid, vitamin, and mineral mix derived from cold-water marine animals, and utilize said mixture to enhance the production of methane from coal by providing nutrition to methanogenic organisms.

It is a further object of this invention to mass produce a nutritious amino acid, vitamin, and mineral mixture derived from cold-water marine animals, and utilize said mixture to enhance the production of antigens; diagnosticums; vaccines; recombinant antibodies; hormones; metabolites; antibiotics; and bacterial products.

### DESCRIPTION OF THE FIGURES

FIG. 1 is a block diagram showing the use of enzymatic protein hydrolysate (EPH) as an additive or as a catalyst in other industrial processes involving cellular or bacterial growth.

FIG. 2 is a block diagram showing the use of EPH as an additive in the food making process.

FIG. 3 shows the use of EPH as a fertilizer additive.

FIG. 4 shows the use of EPH as an additive in the final preparation of animal feed.

FIG. 5 shows the use of EPH as an additive in the final preparation of cosmetic products.

FIG. 6 shows the use of EPH as an additive in the final preparation of externally applied pharmaceutical products.

FIG. 7 shows the use of EPH as an additive in the final preparation of complex bioactive products.

FIG. 8 shows the use of EPH as an additive in the physical, chemical, and biological treatment of wastes, including radioactive wastes and oil pollution.

FIG. 9 is a block diagram showing the use of EPH in the biotechnological processing of petrified coal to produce methane.

FIG. 10 is a block diagram showing the use of EPH as an accelerant in the production of coal bed methane.

FIG. 11 is a block diagram showing the use of EPH as an accelerant in the biotechnological processing of plant biomass into ethanol.

FIG. 12 is a block diagram showing the use of EPH as an accelerant in the biotechnological processing of plant biomass into methane.

FIG. 13 is a block diagram showing the use of EPH as an accelerant in the biotechnological processing of plant biomass into methane and ethanol.

FIG. 14 is a block diagram showing the use of EPH as an accelerant in the production of methane, ethanol, and combustible oils from plant biomass, brown coals, and heavy oil fractions.

FIG. 15 is a block diagram showing the use of EPH as an enhancer of processes that can increase plant biomass and agricultural yield, as well as lower the atmospheric level of carbon dioxide.

FIG. 16 is a block diagram showing the use of EPH to enhance the biotechnological processing of brown coal to make humic acid, minerals, and microelements to produce plant biomass.

FIG. 17 is a block diagram showing the use of EPH to enhance the biotechnological processing of plant biomass and peat to make humic acid.

FIG. 18 is a block diagram showing the use of EPH mixed with brown coal and using a biotechnological process to produce a mixture of humic acid with minerals and microelements, a mixture of amino acids with minerals and microelements, minerals and microelements, and a mixture of humic acids with amino acids.

FIG. 19 is a block diagram showing the use of EPH to enhance the biotechnological processing of plant biomass, animal biomass, peat, and petrified coal to produce a mixture of amino acid and humic acid, a mixture of amino acids and minerals, a mixture of humic acids, minerals, and microelements, and a mixture of humic acid, amino acids, minerals, and microelements.

FIG. 20 is a block diagram showing the use of EPH in a complex technology to enhance environmental clean up, production of fertilizers, increase productivity in agriculture, increase biomass in forests, raise productivity in animal husbandry, transform deserts and infertile ground into fertile locations, raise the quality and quantity of nutritional products for humans, increase biomass for agricultural plants.

FIG. 21 is a block diagram showing the use of EPH to cleanse the environment from pollution and waste destruction, grow plant and other biomass to lower the atmospheric level of carbon dioxide, and produce ecologically uncontaminated energy carriers and energy.

FIG. 22 is a block diagram showing the use of EPH to enhance the biotechnological processing of coal to produce fuels such as methane and the utilization of such fuel to produce electric and other energy with a zero or negative carbon dioxide balance.

FIG. 23 is a block diagram showing the use of EPH to enhance the biotechnological processing of coal to produce fuels such as methane and the utilization of such fuel to produce electric energy with a zero or negative carbon dioxide balance.

FIG. 24 is a block diagram showing the use of EPH to enhance the biotechnological processing of coal to produce fuels such as methane and the utilization of such fuel to produce electric energy and process plant-produced waste with a zero or negative carbon dioxide balance.

FIG. 25 is a block diagram showing the use of EPH to enhance the processing of plant biomass, animal biomass, common waste, industrial waste, and peat and coal to make fuels such as methane, ethane, propane, ethanol, methanol, latexes, etc.

FIG. 26 is a block diagram showing the use of EPH to enhance animal, plant, fungus, and bacteria-derived protein mass in production processes to make an EPH-enhanced food.

### DESCRIPTION OF THE PREFERRED EMBODIMENTS

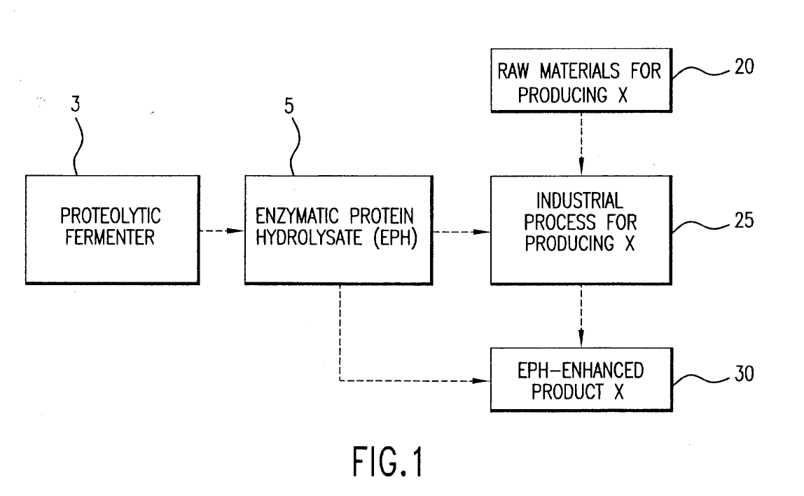

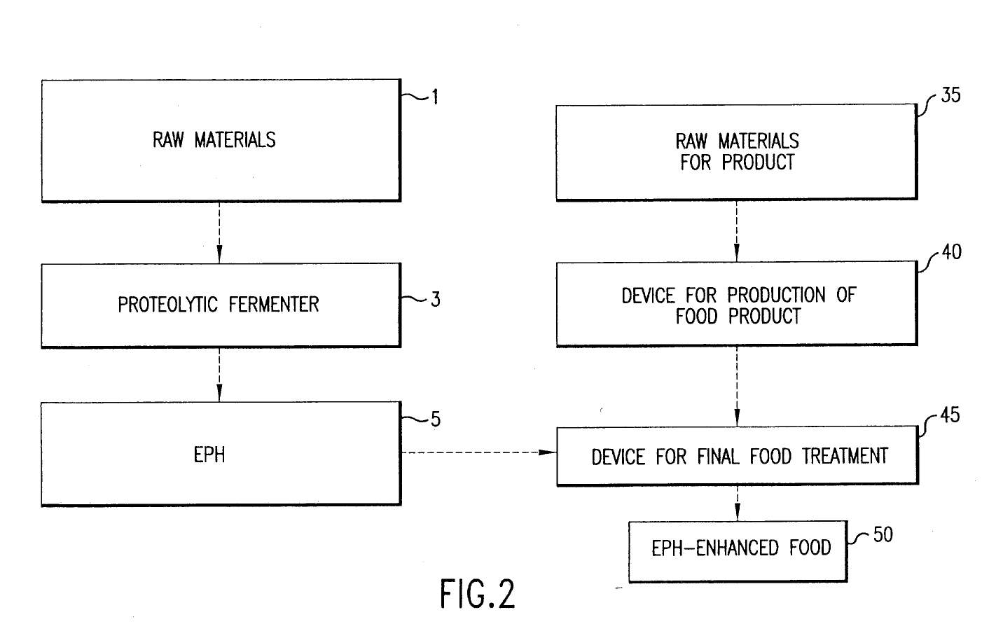

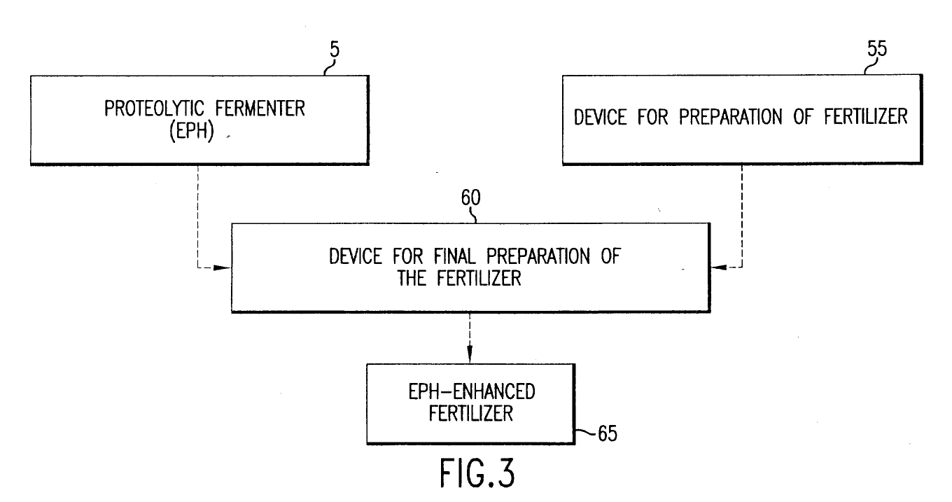

Referring to FIG. 1 and FIG. 2, proteolytic fermenter 3 (sometimes refered to as a bioreactor) produces, through “profound enzymatic protein hydrolysis” of cold water animals, a proprietary enzymatic protein hydrolysate (EPH). Our EPH contains approximately 70%-90% free amino acids, as itemized below. An alternate mixture of EPH may also contain Tryptophan, OH-proline, OH-lysine. The EPH also contains approximately 10%-20% highly molecular peptides. In addition, the EPH contains approximately 3%-5% vitamins, minerals and oils. The EPH minerals include cadmium, lead, mercury, arsenic, natrium, potassium, calcium, phosphorous, magnesium, iron, zinc, copper, manganese, selenium, cobalt, molybdenum, chromium. The vitamins in the EPH include thiamine, riboflavin, pantothenic acid, pyridoxine, nicotinic acid, biotin, folic acid, cyanocobalamine, meso-inosite, vitamin E. For each amino acid below, their effect on the functioning of various organs, tissues and systems of the human body, as well as the possible uses of the amino acids in the treatment of specific diseases are briefly described, as follows:

Lysine—C₆H₁₄N₂O₂—is an Essential Amino Acid.

Promotes the adequate absorption of calcium by bone tissue; participates actively in the synthesis of collagen, which makes up the basis for cartilage and connective and bone tissue; effectively stimulates the synthesis of humoral antibodies and a number of hormones and enzymes. Research has demonstrated that lysine can be effective against the herpes virus, since it stimulates the production of substances that suppress the growth of the virus. A deficiency of lysine in the diet is accompanied by rapid fatigue, an inability to concentrate, irritability, bloodshot eyes, retardation of growth, hair loss, anemia and reproductive problems.

Methionine—C₅H₁₁NO₂S—is an Essential Amino Acid.

Is the main source of sulfur in the cells of the body, necessary for the normal growth of hair, nails and skin; promotes a lowering of the level of cholesterol in the blood; increases the production of lecithin in the liver; reduces the level of fat in the liver and protects the kidneys; a natural gelatinizing (binding) agent for heavy metals; regulates the formation of ammonia in the urine and promotes the formation of urine without ammonia, which reduces bladder irritation; strengthens the structure of the hair and promotes its growth.

Phenylalinine—C₉H₁₁NO₂—an essential amino acid that is used intensively in the brain for the synthesis of norepinephrine—a chemical agent which serves for the transmission of signals between both nerve cells and brain cells; promotes physical activity, reduces hunger, acts as an antidepressant and improves memory.

Threonine—C₄H₉NO₃—an Essential Amino Acid.

Is an extremely important structural element of collagen, elastin and the proteins that make up tooth enamel; prevents the fatty degeneration of the liver; promotes the normal functioning of the gastrointestinal tract.

Leucine—C₆H₁₃NO₂—essential fatty acid that is a decisive component in the synthesis of extremely important biologically active compounds responsible for the generation and transfer of energy in all the cells of the body; stimulates the working of the brain.

Isoleucine—C₆H₁₃NO₂—essential fatty acid that is decisive in the synthesis of extremely important biologically active compounds responsible for the generation and transfer of energy in all the cells of the body; stimulates the working of the brain.

Arginine—C₆H₁₄N₄O₂—a nonessential amino acid which strengthens the immune response to viral, bacterial and parasitic infections; promotes the healing of wounds and the regeneration of tissues of the parenchymatous organs; inhibits the growth of tumor cells; stimulates the synthesis of growth hormone.

Tyrosine—C₉H₁₁NO₃—a Nonessential Amino Acid.

Transmits impulses from nerve cells to brain cells; helps to overcome depression; improves memory; promotes concentration; supports the healthy functioning of the thyroid gland, the adrenal glands and the pituitary gland.

Glycine—C₂H₅NO₂—a Nonessential Amino Acid.

Promotes the delivery of oxygen and energy in cellular synthesis; is responsible for the strength of the immune response; stimulates the synthesis of a number of hormones.

Serine—HOCH₂—a Nonessential Amino Acid.

Responsible for the full assimilation of glucose by the liver and the muscles; strengthens the synthesis of humoral antibodies; participates in the synthesis of the fatty acid sheath around the nerve fibers.

Glutamic Acid—C₅H₉NO₄—a nonessential amino acid which is considered “brain food”; improves mental abilities; helps with fatigue; speeds up the healing of wounds; is used in the treatment of schizophrenia, alcoholism and diabetes mellitus.

Aspartic Acid—C₄H₇NO₄—a nonessential amino acid. Promotes the excretion of ammonia from the body, since ammonia is a highly poisonous substance for brain cells. Recent research has demonstrated that aspartic acid increases resistance to fatigue and improves endurance.

Taurine—C₂H₇NO₃S—a nonessential amino acid which is formed from cysteine in the liver or from methionine in other organs. It is present in milk and in some sea animals. Taurine participates in the regulation of the process of stimulation in the central nervous system; it has an antiarrhythmic effect on the cardiac muscle; it is a factor in the control of many of the biochemical reactions that take place in the process of aging; it promotes the neutralization of free radicals that appear in the cells of the body from environmental factors; it inhibits aging processes; it binds free radicals; it neutralizes toxins; it is an active participant in the restoration of epidermal tissue damaged by burns or by transplants or other surgical effects. The hair and skin contain up to 14% cystine.

Histidine—C₆H₉N₃O₂—a nonessential amino acid. Found in abundance in hemoglobin; widely used in the treatment of rheumatoid arthritis, allergic diseases, ulcers and anemia. A histidine deficiency is a cause of poor hearing.

Alanine—C₃H₇NO₂—a nonessential amino acid which is an important source of energy for muscle tissue, the brain and the central nervous system; strengthens the activity of the immune system and improves the humoral immune response; promotes the metabolism of sugar and organic acids.

Proline—C₅H₉NO₂—a nonessential amino acid. It is the main component of all connective tissue and is necessary for the normal functioning of the joints, tendons and ligaments; supports the working fitness of the cardiac muscle.

Ornithine—C₅H₁₂N₂O₂—a nonessential amino acid. It has not been isolated from proteins except after alkali hydrolysis.

Description of Complex Technologies Using EPH

Complex technologies imply the presence of two or more technological processes, united by the parameters of solving an existing problem. Enzymatic protein hydrolysate (hereinafter “EPH”) can be characterized as a highly molecular fish protein. It contains a mixture of approximately 70%-90% free amino acids, 10%-20% highly molecular peptides, and 3%-5% minerals, vitamins and oils. EPH may be an element to be included with various devices and various processes as complex technologies because of its properties as a complete source of cellular nutrition for microbes, including bacteria, fungi, yeast, eukariotic organisms and archaebacteria.

For instance, the complex technology for waste processing can contain several levels of processing-physical, chemical, biological. Also some parallel chains may be contained-processing of common, industrial, plant, and animal wastes. All such technological chains are interrelated when they are biologically based. EPH facilitates a maximally effective and balanced development of any such biological process.

### EXAMPLE 1

### The Complex Generic Technology For Converting Raw Materials in Biological Industrial Processes Into Products That are EPH-enhanced

Biological industrial processes can be enhanced by the addition of EPH into the biological organisms' reproduction phase, growth phase, and/or decline phase in any process including the production of: antigens; diagnosticums; vaccines; recombinant antibodies; hormones; metabolites; antibiotics; bacterial products; amino acids; products for parenteral and enteral nutrition; bacterial biomass; yeast biomass; fungal biomass; seaweed biomass; kelp biomass; biomass of all advanced plants; methane; hydrogen; biodiesel fuel; ethyl alcohol; methyl alcohol; prophyl and other alcohols; oil substitutes (latex); methanol; ethanol; gasoline from long chain hydrocarbons; biodiesel from waste grease; coal bed methane; kerosene; bread; cheese; kefir; yogurt; sour cream; vegetable oils; animal oils; animal and vegetable fats; glucose; fructose; sucrose; dextrose; organic acids, such as lemon, vinegar, orange, etc.); syrups; juices of fruits and vegetables; wine beer; cider; spirits; brandy; protein or unicellular organisms; mushroom protein; vitamins; minerals; fool colorants; flavorants; margarine; edible vegetable-derived pastes; edible animal-derived pastes; food thickeners; meat preserves; fish pastes; fish preserves; poultry pastes; vegetable preserves; fruit preserves; spices for food preparations; solvents, including acetone, butanol, isopropanol; ferments, including proteinase, glucoisomerase, amylase, hydrolase, lastase, etc.; biopolymers; bioextractive metallurgy for uranium, gold, silver, copper, zinc, cobalt, rare earth minerals, coal, platinum; mercury, arsenic, selenium, gallium, titanium, molybdenum, chromium, etc.; xenobiotics, fertilizer; humic acid; fulvic acid; and biological processes for treating common wastes, plant wastes, animal wastes, industrial wastes, peat, coal, lignite, petroleum, chemical wastes, combustible slate, hydrocarbon shales, methane ice in the seabed; remediation of biological materials; remediation of chemical materials; remediation of toxic wastes; etc.

### EXAMPLE 2

### The Complex Technology For Producing Higher Quality Food Products By the Addition of EPH

It is primarily a quantity of peptides of various sizes and free amino acids which are formed in the hydrolysis of proteins. Hence any hydrolysate represents a mixture made up of free amino acids and peptides. In addition, in this mixture there are simple sugars and polysacharrides, fragments of nucleic acids, vitamins, minerals and trace elements. In the use of hydrolysates for food, food supplements, feed supplements and nutrient media for cells and microorganisms, the peptides act as sources of amino acids, of nitrogen, carbon and sulfur. In this process, peptides as a source of amino acids are typically more effective than synthetic mixtures.

F. J. Sussman and C. Gilvard (1971) have established that bacteria use polypeptides more actively than free amino acids. The authors explained this phenomenon by the existence of independent transport for polypeptides. Thus, according to data of K. A. Pittman, et al. (1967), Bacteroides Ruminicola do not use free proline from the medium but rather consume proline-containing peptides. Similarly, scientists have observed faster intake of peptides, as opposed to free amino acids, in the small intestine (R. F. Grampton, 1970), as well as faster intake of the products of enzymatic hydrolysis as compared to the products of acid hydrolysis of the same proteins or as compared to mixtures of free amino acids (D. B. A. Silk, et al., 1973). Amino acids pass through cell barriers against the concentration gradient, which attests to the presence of active transport in the intestine. Many authors have observed phenomena of competition for these transport systems in this process. As H. Newey and D. Smith (1962) hypothesized, and as has been confirmed subsequently, peptides and amino acids are transported in the epithelium of the intestine by different mechanisms in different sections of the small intestine. Hence it has been established for both higher animals and for microorganisms that there are specific mechanisms for the assimilation and transfer of peptides. In man and animals there are individual sections of the intestine through which peptides are absorbed. Microorganisms have their own permeases for passage through cell walls. As a result, peptides are more easily assimilated.

Biologically active peptides are contained in various organs and tissues and are formed in natural biological processes in the blood and in the gastrointestinal tract. It is known that peptides play a mediator role among the regulatory systems of the bodies of higher animals: the nervous, humoral and immune systems. They are material carriers of information and have a regulatory effect of their own. Hundreds of peptides, each of which is characterized by polyfunctionality in its effects (I. P. Ashmarin, 1985), have been identified. Among the regulatory peptides, a few functional groups can be distinguished. These groups are: peptide hormones produced by the organs of the endrocrine system; tissue hormones—kinins; and a large group of peptide immunomodulators (immunohormones), as well as immunomodulators and mediators of exogenous origin (F. I. Ershov and V. V. Malinovskaya, 1996). The hormones secreted by the endocrine glands—the thyroid and parathyroid glands, the pancreas and the adrenal glands and gonads—“maintain” the organs that produce them; the hormones of the pituitary gland regulate the activity of the other hormones. The structure of most of them was studied long ago. They have the form of linear or branched peptides, mainly of small sizes. For example, the oligopeptide hormones of the pituitary gland have molecular weights of 1500-6000 D and are made up of 13-39 amino acids.

In addition to these hormones, there are other very active peptide regulators, for the production of which there are no special organs. They are produced by the cells of various tissues and are known as kinins. The kinin hormones are formed from inactive precursors in the blood plasma or in other systems which are not strictly localized. Bradykinin and angiotensin-linear peptides containing 8-9 amino acid radicals-are examples of kinins. Gastrin and secretin, which are produced in the digestive tract, can be considered kinins, as can the biologically active peptides of the thymus. The thymus peptides, however, are more often considered as immunomodulator peptide regulators of the immune system, or immune system hormones (R. V. Petrov, 1987). The communication among the various systems of the body and the regulation of the processes involved with immunity can be accomplished, on the one hand, by mediators produced by the cells of the immune system (immunoglobulins, lymphokines, interleukins, interferons, etc.) or, on the other, by neuropeptides of the central nervous system. A group of regulator peptides (myelopeptides) which carry on the transfer of information between systems have been extracted from the bone marrow. The peptides in question, which have molecular weights of 1300-2000 daltons, stimulated the production of antibodies at the peak of the immune response and possessed analgesic and opiate-like effects (A. A. Mikhaylov, et al., 1985). The term “cytomedins” has been proposed to designate the peptide mediators that specifically regulate the processes of development and interaction of cell populations (V. G. Morozov, et al., 1983). It has been established that theses peptides, which are primarily of a basic nature and have molecular weights of 1000 to 10,000 daltons, have tissue-specific effects and are responsible for the processes of proliferation and differentiation of cells. The amino acid compositions of practically all the known biologically active peptides of natural origin have now been studied.

The regulatory systems of the body-the endocrine, kinin, immune and central nervous systems-are closely connected to each other and are regulated by identical structural units or structural units constructed according to the same type, which are active centers of polypeptides and proteins or low molecular weight peptides formed from precursors (immunoglobulins, interferons, thymus hormones, kinins, histocompatibility proteins) in organic proteolysis reactions (G. A. Chipens, et al., 1985). It is suggested that each molecule of a peptide-protein bioregulator (effector) has specific active centers that provide for its interaction with cells of the immune system, the central nervous system and other systems. The absence of the appropriate receptors results in insensitivity of the cells to the effects even of large doses of an effector (S. I. Kusen, et al., 1985). Based on the idea of maximum versatility of the mechanisms used in nature, one can hypothesize that the amino acid sequences of cell receptors can be the same, that different bioregulatory systems can have the same structure, and that the differentiation of receptor systems is accomplished at the level of the conformation of mediator molecules. It is known that the classical neuromediators are low molecular weight peptides with a molecular weight of the order of 200 daltons, while immunomodulators are proteins or high molecular weight polypeptides. In this case, universality of language can be realized if the information carrier is not a whole molecule but only a fragment of the molecule which is equivalent in size to the neuromediators. Such molecules can be formed as a result of limited proteolysis at the level of cooperation of macrophages and lymphocytes or ligand-receptor complexes in the interaction of mediators with receptors. Limited proteolysis can occur immediately at the surfaces of the interacting cells during the cooperation of the cells in an immuno—(neuro)—synapse, or in intracellular vacuoles that appear in the process of endocytosis of immune or other ligand-receptor complexes. The components of these complexes can include immunoglogulins, interleukins, monokines, proteins of the histocompatibility system and other immunomodulators and mediators. It is suggested that the products of this kind of hydrolysis, which perform a bioregulatory function directly, be called “tetins” (from the French “tête-à-tête”). Hence, if the hormones are regulators of overall effects, and kinins are regulators of local effects, tetins can be considered as spot regulators. Consequently, the tetin principle should operate throughout all the regulatory systems. This kind of spot mechanism for the effects of biologically active peptides would seem to conform to the greatest degree to the nature of their interaction with the systems of the body. In the formation of ligand-receptor complexes, in which the ligand is a biologically active compound, and the receptor is a structure on the surface of sensitive cells, the size of the ligand molecule should be limited. The optimum size for an active peptide is 6-8 amino acid radicals, of which 3-4 radicals made up the active center proper, which is complementary to the receptor. On the other hand, the ligand chains can be of larger sizes but can form loops that contain an active center-which is still made up of 3-4 amino acid radicals.

It has come to be known as a result of studies by a number of researchers (Keay, 1975; Jenkin, Yang and Anderson, 1979, and others) that biologically active peptides are also formed in the enzymatic hydrolysis of proteins conducted in vitro. For example, after the limited hydrolysis of casein with the use of pepsin, it was possible to isolate a glycomacropeptide that was identified as a fraction of biologically active peptides which act as strong inhibitors of gastric secretion and the motor system. The physiological role of inhibition apparently is a matter of restricting proteolytic activity for the purpose of preserving biologically active proteins (gamma-globulins, lactoferrin, lysozyme) and peptides for the body against degradation and inactivation. In the process of proteolysis of beta-casein, a peptide is released which conforms to the sequence 60-66 and which is called beta-casomorphine; it possesses opioid and analgesic effects: Tyr-Pro-Phen-Gly-Pro-Ile (E. Y. Stan, 1987). Hexapeptide 54-59 of the following composition was extracted from beta-casein from mother's milk after proteolysis, purified and sequenced: Val-Gly-Pro-Ile-Pro-Tyr. The hexapeptide exhibited immunostimulating and antibacterial activity, presumably due to its stimulating effect on macrophages. From pepsin hydrolysate of cow casein, an opioid-type peptide called exorphin was extracted with the sequence 90-96 (Arg-Tyr-Gly-Tyr-Leu-Gly) (C. Zioudron, et al., 1979; S. Loukas, et al., 1983). Similar peptides are formed under the effect of caseinolytic microorganisms in the production of certain types of cheese and other sour milk products. Hence the biologically active peptides which are formed in the process of the digestion of various proteins take part in the saturation mechanism, have an impact on the gastrointestinal tract, stimulate the immune system and perform other functions, as a result of which they have been called “food hormones” (J. E. Morley, 1982).

The biological effect of peptides on the growth of microorganisms was established during the 1940's. H. Sprince and D. W. Woolley (1944-1945) discovered a factor of peptide nature in a number of products of natural origin (liver, flour, tomato juice, yeast, etc.) which stimulates the growth of certain microorganisms and called it streptogenin (E. Schroeder and K. Luebke, 1969). As was determined in further research, trypsin hydrolysates of various proteins have streptogenin activity that varies in regard to the strength of the effect Insulin hydrolysate possessed the greatest activity in the experiments in question. It was demonstrated later (E. R. Stadtman and T. C. Stadtman, 1953) that arginine-containing peptides are significant for a culture of E. rhusiopathiae. The presence of tyrosine peptides in the medium is required for S. faecalis; free tyrosine cannot support the growth of the microbes. It was established, in addition, that while low molecular weight nutrient substances (amino acids and others) are necessary for the multiplication of the bacteria, peptides are effective for toxin formation.

Biological industrial processes for the production of food can be enhanced by the addition of EPH into the biological organisms' reproduction phase, growth phase, and/or decline phase. High-quality EPH, containing high percentages of free amino acids, which has good solubility in water and possesses a pleasant taste, is the best product of all the products available for the growth of muscle mass; therefore, it is of particular interest as a daily dietary product for all athletes, both amateur and professional. Physicians, physiologists and trainers have proven convincingly that if muscle tissue is not supplied with amino acids and vitamins, it will not grow, despite regular and intense training. The natural, balanced makeup of the EPH in regard to essential and nonessential amino acids and vitamins makes it possible to classify it as a unique source for easily accessible “building materials” for the restoration and growth of muscle mass. EPH is a very compact, high-energy food. This product takes up much less space in transportation and storage than any other food that is comparable to it in quality and nutritional value. Thus EPH is a special requirement as “extreme food” for astronauts, mountain climbers, tourists, sailors and soldiers.

This product can also be considered the ideal “emergency supply” to be collected and stocked by every country for cases of natural disasters and other catastrophes. At the same time, EPH is an independent “superfast” food for everyone suffering from protein deficiency in areas of environmental disasters, mass migrations of the population, catastrophes and wars.

EPH is a unique nutritional supplement for infants due to its uniquely balanced amino acid composition supplemented by the entire B-vitamin complex and vitamin E, as well as other vitally necessary minerals and trace elements. This product is capable of being taken up immediately in metabolic processes, thus promoting optimum growth and development of the child's body.

It is common knowledge that modern food products produced by so-called “continuous industrial processes” have a pronounced deficiency of essential amino acids, vitamins and trace elements. It is primarily children who suffer from this deficiency. The addition of EPH to various types of food products intended primarily for feeding children and adolescents will make it possible to compensate fully for the deficiency.

With age, especially in old age, senile changes build up in the body; these changes are manifested primarily in the poor (incomplete) assimilation of a normal, quite nutritious diet. Impaired digestion and a reduced capacity to assimilate the necessary nutrients from the diet result in poor health from vitamin deficiency and muscular dystrophy in the elderly, even when they have a nutritious, high-quality diet. No one has made a serious attempt until now to develop special food for elderly people to help them maintain their quality of life for a long time.

EPH is the ideal foundation for such a diet. When this product is used, the body does not need to digest it: it absorbs it from the gastrointestinal tract directly into the blood. As a result, EPH represents an absolutely real means for prolonging life. All that a human consumes as nutrition is plant and animal products, as well as the output of bacterial and fungal activity. In summary, humans consume proteins, fats and carbohydrates, microelements, minerals, and vitamins, as well as some bioactive substances. All of these ingredients of the human diet may originate form any source. EPH will accelerate production of these components, as it is incorporated into these complex technologies.

One example is production of protein for nutrition. Proteins may be obtained from animals, plants, bacteria and fungi. The protein may either be a product in itself, or it may be contained in meat, milk, produce, mushrooms, etc. In each of the cases, there will be unique interaction between EPH and other parts of the process. For instance, to increase the effectiveness of animal husbandry, EPH must support plant (animal feed) growth, as well as the growth of the animals themselves, creating amino acidic supplements for their diet and so forth.

The use of EPH not only increases the growth rate of protein-containing organisms, but also accelerates the production of protein-filled foods as it stimulates those parts of the processes that require biotechnological methods (production of cottage cheese, cheese, sour cream, yogurt, etc., obtaining pure proteins from soy, bacteria and fungi). The EPH can also stimulate the activities of specialized bacteria that produce amino acids. Moreover, as the food is processed further, the EPH can facilitate the addition of amino acids into the product.

In the case of carbohydrates, EPH can increase the growth rate of the substrate, as well as the production process for carbohydrate-filled foods (production of beer, wine, cider, various sodas, bread, etc. Furthermore, the use of EPH allows for addition of amino acids into the carbohydrate-filled food at the final step of its preparation, to increase its nutritional value and for any medicinal purpose.

As for production of fats, we find the same statements to be true. EPH can accelerate the growth of lipid-containing organisms, influence the production process of lipids, and can facilitate the addition of amino acids to the final product.

Naturally, it is a rare case where a strictly protein-filled, carbohydrate-filled, or lipid-filled foods are produced. Usually, we consume complex products that contain proteins and fats or fats along with proteins and carbohydrates, or fats and carbohydrates or all of the above plus vitamins and minerals, etc. Therefore, we can establish a complex technology of nutrition using EPH, which contains the three processes described above. Whether together or separately, these processes need not be executed in strict accordance with FIG. 2. Possible options include preparation of a single product from several substrates or of several products given a single substrate, or using one substrate to prepare a single product. The products as well as substrates may be complex and have numerous ingredients, as well as be simple. Such products may include vegetable oil, soy protein, or fructose, and the substrates may be soy, sunflower, sugar beets, etc.

EPH may enhance processes to produce a stable liquid diet composition has been described in U.S. Pat. No. 4,497,800, by Larsen and Reyes, which is hereby incorporated by reference in its entirety. It is not proven whether combining EPH with the process described in U.S. Pat. No. 4,497,800 will be suitable for the liquid diet.

### EXAMPLE 3

### The Complex Technology For Converting Plant Biomass While Increasing Agricultural Yield, and Reducing Atmospheric Levels of CO₂

This complex technology is based on biotechnological processing of various input materials with productions of fertilizers as the goal. Input materials may consist of plant and animal biomass, common and industrial waste, peat, various types of coal, various types of oil and its fractions, various other natural fossil fuels or chemical substances and materials. This complex technology can be utilized as a single unit, or only one part of it. One example is the biotechnological processing of brown coal and humic acid, minerals, and microelements using EPH. The EPH acts as an accelerant of microbial activity by providing complete cellular nutrition. Stronger, healthier microbes will act more quickly and efficiently to degrade plant biomass if introduced in the growth phase. Another possible option is using several sources of input material to obtain one type of fertilizer, for example biotechnological processing of plant biomass and peat into humic acid.

The process to produce a potassium polyphosphate protein hydrolsate fertilizer has been described in U.S. Pat. No. 4,491,464, by Ashmead, which is hereby incorporated by reference in its entirety. It is not proven whether combining EPH with the process described in U.S. Pat. No. 4,491,464 will enhance the fertilizer's effectiveness. The method for increasing fertilizer efficiency is described in U.S. Pat. No. 5,840,656, by Kinnersley, et al, which is hereby incorporated by reference in its entirety. It is not proven whether combining EPH with the process described in U.S. Pat. No. 5,840,656 will enhance the fertilizer's effectiveness.

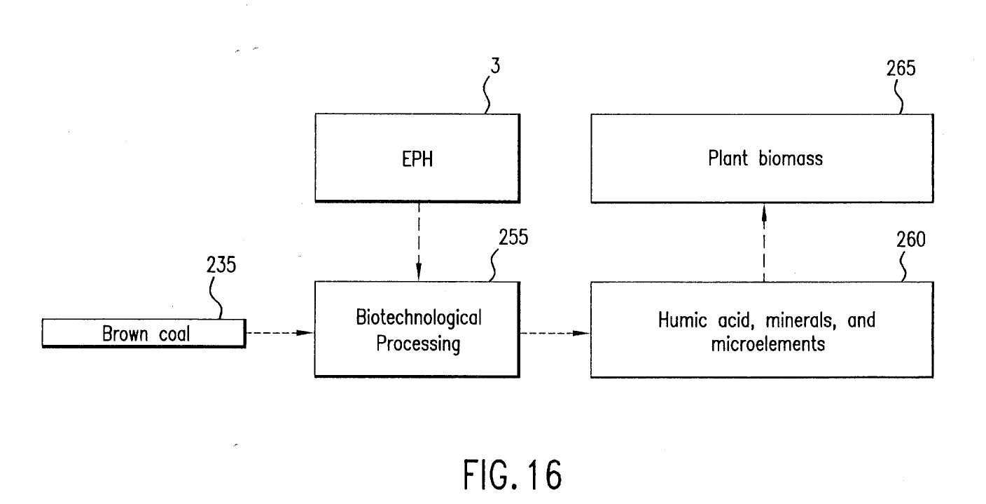

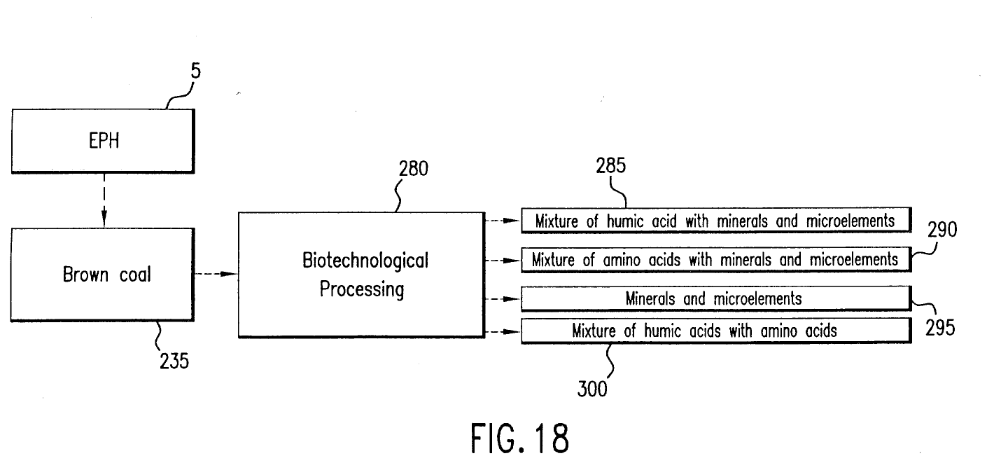

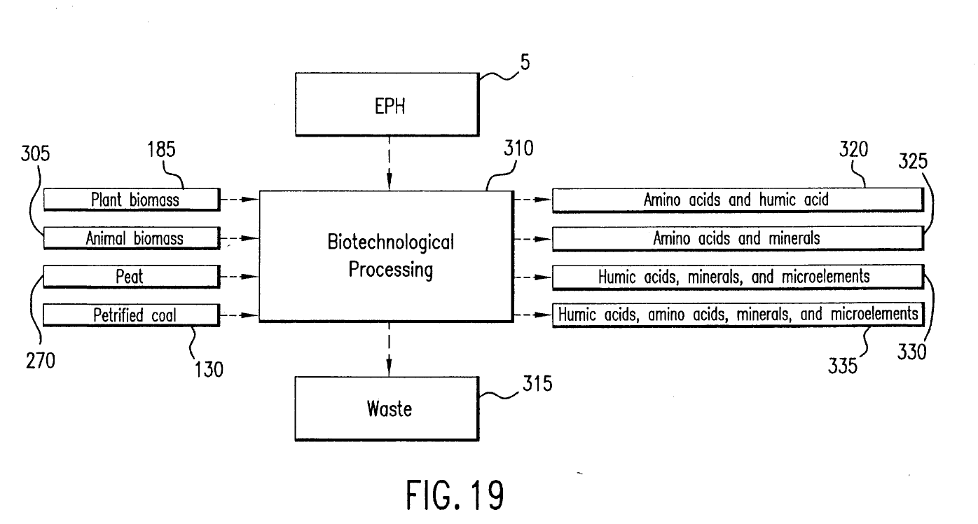

It is also possible to obtain several types of fertilizer from one input source. An example is production of fertilizers, all from brown coal. Referring to FIGS. 16 to 19, these can include: mixture of humic acid with minerals and microelements; mixture of amino acids with minerals and microelements; minerals and microelements, and; mixture of humic acid with amino acids.

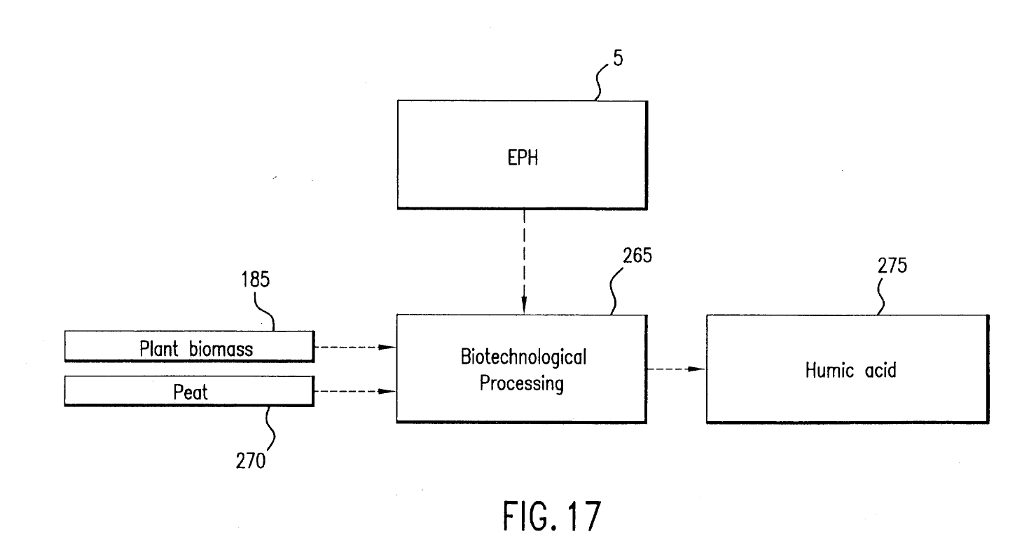

Finally, there is also the option of using several input sources to produce several types of fertilizer. For instance, using plant biomass, animal biomass, petrified coal and peat to obtain fertilizers. Referring to FIG. 17, these can include the following mixtures: amino acids and humic acid; amino acids and minerals; humic acid and minerals and microelements, and; humic acids, amino acids, and minerals and microelements.

Of course, the complex nature of this technology will affect no only the types of possible input materials and varieties of fertilizers obtained, but also the types of effects this process can have on the environment. The use of fertilizers increases the quantity of plant biomass, which is extremely useful in arid and infertile locations. Naturally, the use of fertilizers also increases the productivity of agricultural plants and feed grasses, thereby also increasing the productivity of animal husbandry. All of these factors influence the quantity and quality of nutritional products available to humans. Moreover, an increase in biomass is very important in terms of lowering the atmospheric level of CO₂ and fighting the greenhouse effect. The human race has not yet invented a method of lowering CO₂ superior to that used by nature itself-growth of plant biomass.

### EXAMPLE 4

### The Complex Technology For Enhancing the Food Value of Animal Feed By the Addition of EPH

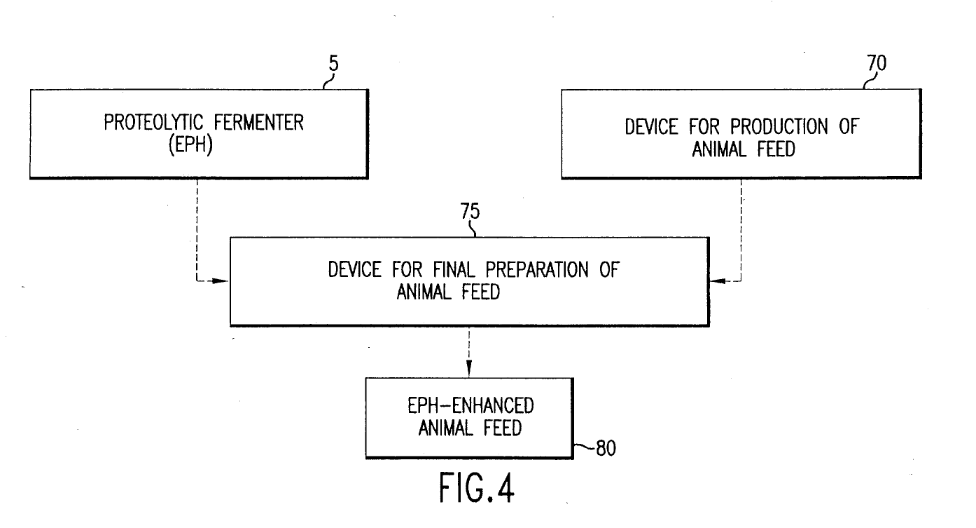

Referring to FIG. 4, this complex technology is based on biotechnological processing of various input materials with production of animal feeds as the goal. Input materials may consist of plant and animal biomass, food supplements, fillers, and other components. The animal feed industry is a mature industry with constantly improving research on the best products to include for animal nutrition. Because of the complete mix of amino acids, peptides, vitamins and minerals contained in EPH, it enhances the quality of the nutrition for any animal. In particular, the highly accessible nature of the free amino acids makes them easily absorbed and utilized by animals with less digestive processing than for normal feed materials.

The quality of fish protein produced by the proteolytic fermenter is so high that a significant amount of materials suitable for use as pet foods can be sold. The quality levels produced by the core technology can be used to suit a particular market. For example, the quality level for pet fish is not the same as for dogs or cats. Higher quality fish protein can easily be used as animal feed for omnivores. Additionally, the EPH can be used as a dietary supplement to improve the health and physical condition of virtually any animal. EPH can be added to pellets or other forms to provide highly nutritional feed in the form most suitable for ingestion for any type of animal, including fish, birds, mammals, reptiles, and marsupials.

### EXAMPLE 5

### The Complex Technology For Enhancing the Enzymatic and Nutritional Properties of Cosmetics By the Addition of EPH

Changes in the body with age, impairment of protein nutrition, deterioration of health due to vitamin deficiencies and disruption of the supply of minerals and trace elements are reflected primarily, as in a mirror, in the condition of a person's skin. An enormous number of cosmetics are intended to hide the changes in the skin that occur with aging. A considerably smaller number of cosmetic are intended to nourish the skin and maintain the normal physiological condition of the skin. Frequently, however, products are introduced into the formulas for cosmetics which are not capable of penetrating the epidermis, but which remain on the surface, thus blocking the natural respiration of the body through the skin. The task of modem therapeutic cosmetics is to develop cosmetic preparations with a pronounced “nourishing effect” for the skin cells-the keratinocytes.

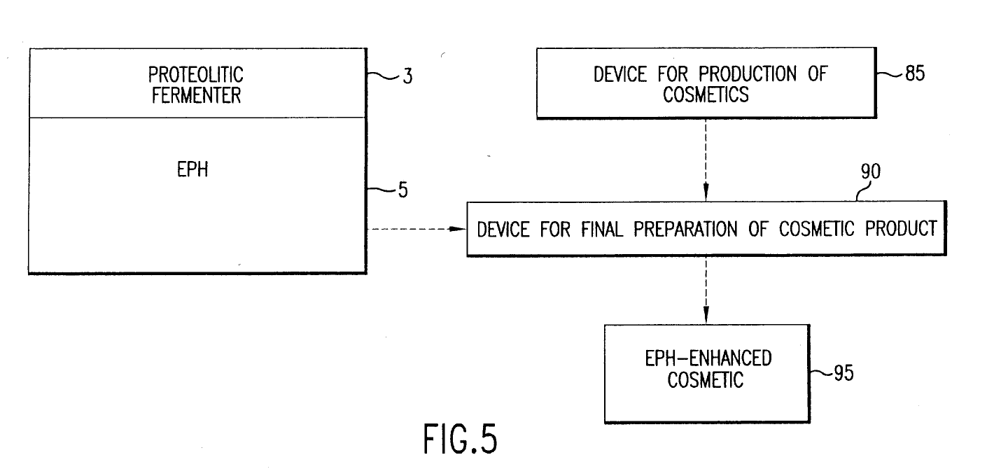

Referring to FIG. 5, addition of EPH to cosmetic creams and lotions is an excellent mechanism to provide direct, local nourishment for the skin cells. The free amino acids, biologically active peptides, vitamins, minerals and trace elements from the EPH are capable of penetrating freely into the skin cells and the space between cells. Such creams and lotions actually stimulate the normal reproduction and vital activity of skin cells. Cosmetic creams and lotions containing EPH have the purpose of preventing many of the negative effects on human skin from both natural and artificial environmental factors.

The skin of the head often suffers both from the harmful effects of the environment and from cleansing agents of poor quality. These effects cause seborrhea (flaking of the skin) or hair loss (balding). Shampoos that contain EPH are intended primarily to protect the skin of the head against the harmful environmental factors. Free amino acids and biologically active peptides, in turn, nourish hair follicles directly through external application, thus assisting in growth and vitality of the cells.

### EXAMPLE 6

### The Complex Technology For Enhancing the Production of Pharmaceutical Products and Preparations

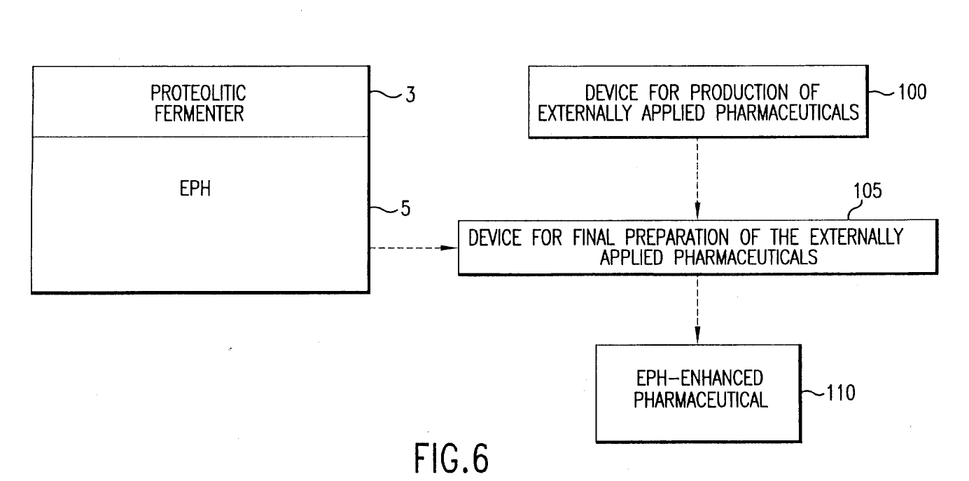

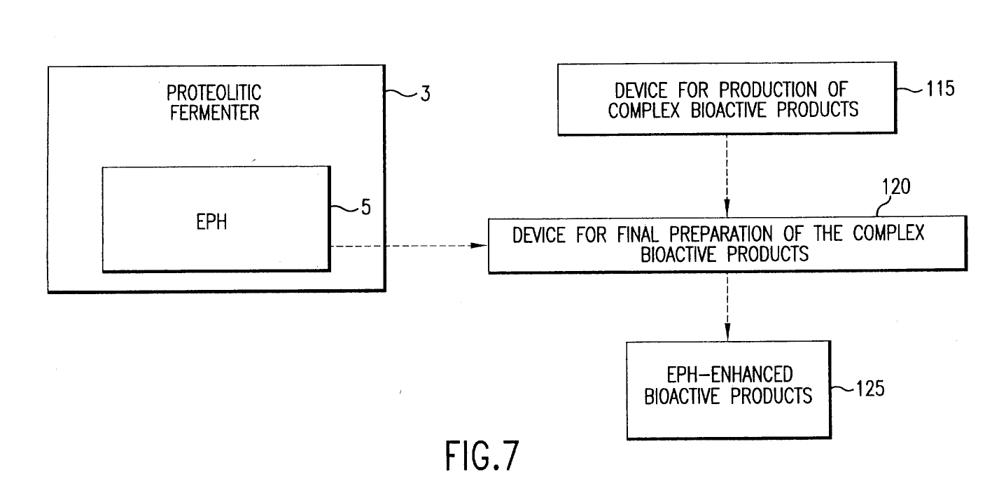

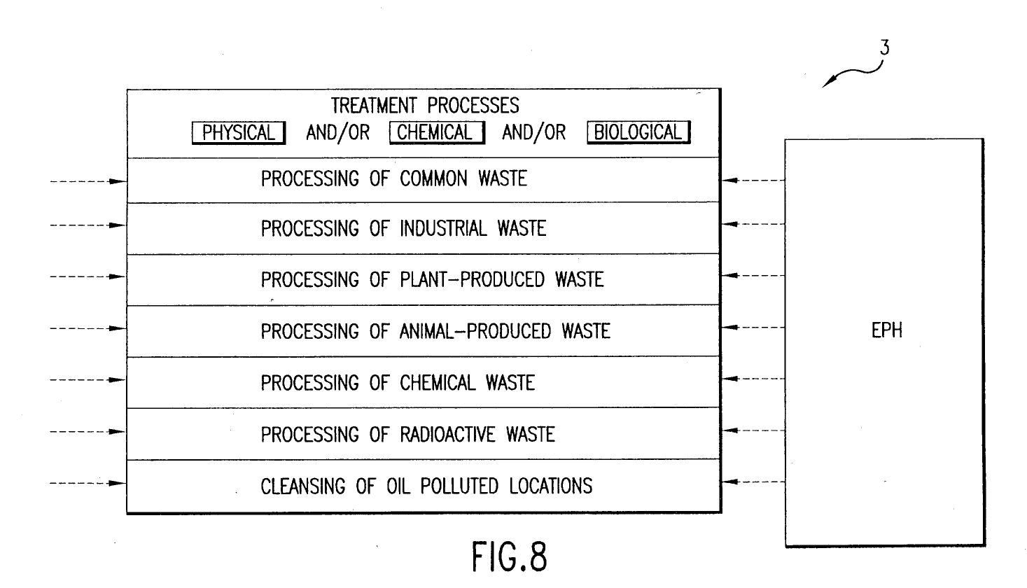

Referring to FIG. 6, addition of EPH into the reproduction phase, growth phase, and/or decline phase of biological organisms that produce pharmaceutical products will enhance the speed and efficiency of the production process. There are a multitude of pharmaceutical products and methods to produce them. In most cases, the utilization of EPH can increase the rate of production, increase the quantity produced, reduce the time used by the process, enhance product quality and boost its effectiveness.

EPH can positively influence the production of a multitude of input materials that are utilized either as pharmaceuticals or as the input material from which pharmaceutical products are refined or extracted. EPH may also influence the production of the pharmaceutical item itself. One example is the production process of an antibiotic. EPH can directly influence the rate and quality of production, stimulating the activity of fungi that produce it. Moreover, many pharmaceutical items nowadays are produced using bacterial activity-hyperproducents. EPH can accelerate the bacterial activity and thus the production process itself. In a score of cases, a particular reaction is required to produce a pharmaceutical item using the input materials. This particular reaction can be catalyzed by EPH or the input materials for this reaction may be obtained using EPH. A multitude of such methods and technological chains is possible, but all of them will share certain features-the influence of a proteolytic fermenter to produce EPH that is then utilized in the direct production of the pharmaceutical item.

Furthermore, the product of a proteolytic fermenter—amino acids, short bioactive peptides, lipids, minerals, microelements, vitamins, glycogen—can all be a part of the final product. They can be included in the product all together, separately, or in any combination. The mechanism of this complex product's activity can be of any nature—a general nutritional supplement with amino acids to nourish the body, or a complex medication for trauma, heart attacks, strokes, psychological disorders or any other illnesses. There can be a wide variety of such complex products, but all of these cases can be united under the term, the use of a proteolytic fermenter to create a complex pharmaceutical product by uniting a given medication with products of the activity of EPH.

The process to create antibiotic compounds from certain bacteria has been described in U.S. Pat. No. 4,448,714, by Cunliffe, et al, which is hereby incorporated by reference in its entirety. It is not proven whether combining EPH with the process described in U.S. Pat. No. 4,448,714 will accelerate bacterial growth or increase antibiotic production.

Additionally, EPH can be used directly to produce the pharmaceutical item, since the fermenter's product can be used to achieve this aim. Examples include a mixture of amino acids to supplement the body, nourish the ill during the post-operative period, and a mixture of some protein-specific or organ-specific amino acids to deal with some particular illnesses—for instance dealing with the consequences of brain trauma using a protein hydrolysate from brain tissue, or dealing with a heart attack using a protein hydrolysate from the heart's muscle tissue, or dealing with schizophrenia or manic depressive psychosis using a specific array of deficient amino acids.

In a number of cases, the product is made of the by-products of the proteolytic fermenter's activity can be supplemented with various ballasts or enhancing substances or both. One example is a complex nutritional product for diabetics, including an array of amino acids, fibers, minerals, microelements, vitamins, lipids, and highly molecular carbohydrates. There can be a multitude of such products, but all of them can be united under the term, the use of by-products of EPH as a pharmaceutical product or for the direct creation of a pharmaceutical product based on these by-products.

From a technological standpoint, all of the abovementioned mechanisms of utilization of EPH for producing pharmaceutical items will be completely analogous for the production of medication for animals, plants, bacteria, and fungi. Also, the approach to the production of various nutritional supplements not defined as “medication” will be analogous.

### EXAMPLE 7

### The Complex Technology For Enhancing the Production of Bioactive Materials

EPH has unique properties that make it particularly suitable for preparations for parenteral protein nutrition (highly purified and sterile). In a number of pathological conditions (obstruction of the esophagus, impairment of absorption from the intestine, severe intoxication, acute renal failure, surgery on the stomach, intestine and other internal organs), the need for the parenteral introduction of products that provide for optimum protein nutrition of the body often arises. The parenteral introduction of proteins (bypassing the intestinal tract) results in the development of sensitization, and the repeated introduction of the proteins can lead to anaphylaxis (anaphylactic shock).

To avoid these complications, either a mixture of synthetic amino acids or preparations containing amino acids obtained by the deep hydrolysis of proteins are used for parenteral introduction. Amino acids, contained in high concentrations in EPH, in contrast to proteins, possess neither species nor tissue specificity; hence they cause no side effects when they are introduced, while providing the body with the necessary building blocks for the synthesis of its own tissue structures.

The process to eliminate aspartic acid and glutamic acid from protein hydrolysates has been described in U.S. Pat. No. 4,675,196, by Villa, et al, which is hereby incorporated by reference in its entirety. It is not proven whether combining EPH with the process described in U.S. Pat. No. 4,675,196 will be effective. In addition the process to formulate a nutrition product using amino acids has been described in U.S. Pat. No. 5,547,927, by Cope, et al, which is hereby incorporated by reference in its entirety. It is not proven whether combining EPH with the process described in U.S. Pat. No. 5,547,927 will be effective.

EPH can serve as a complete product for parenteral protein nutrition. For the purpose of the best assimilation of the amino acids by the body, it is recommended that the preparation be included in the formulas for nutrient compositions for intravenous injection or for introduction into the stomach or the small intestine by way of a probe.

EPH can also be used successfully as a therapeutic preparation in diseases accompanied by protein deficiency and, when necessary, for a high-protein diet (in hypoproteinemia and emaciation of the body, including these conditions in the presence of gastrointestinal diseases with impairment of the absorption of proteins, and in blockage of the intestine, intoxication, bum disease, sluggishly granulating wounds and radiation sickness). This preparation can be used extensively as part of a fortified diet for patients for the purpose of improving metabolic and reparative processes during the post-operative period. For the purposes listed above, the preparation can be produced in the form of nutrient solutions, tablets, capsules and suppositories.

The free amino acids, vitamins, biologically active peptides, minerals and trace elements contained in EPH are capable of easily penetrating the mucous membranes and the skin. This makes it possible to classify EPH as a highly effective biogenic preparation for local use in the form of ointments, creams, gels and sprays.

Preparations containing EPH should be used to improve metabolic processes and accelerate tissue regeneration in the presence of trophic ulcers, gangrene, bedsores, burns, radiation ulcers and skin grafts, as well as in various forms of dermatitis.

Preparations containing EPH can be used in a special medicinal form (a 20% gel) in the treatment of diseases of the cornea (trophic epithelial keratitis, infectious corneal dystrophy).

In addition, preparations containing EPH can be applied to surgical incisions to assist in faster healing due to the direct nutrition of the cells by the free amino acids and other nutritional components of EPH.

### EXAMPLE 8

### The Complex Technology For Enhancing the Biological Treatment of Waste Materials

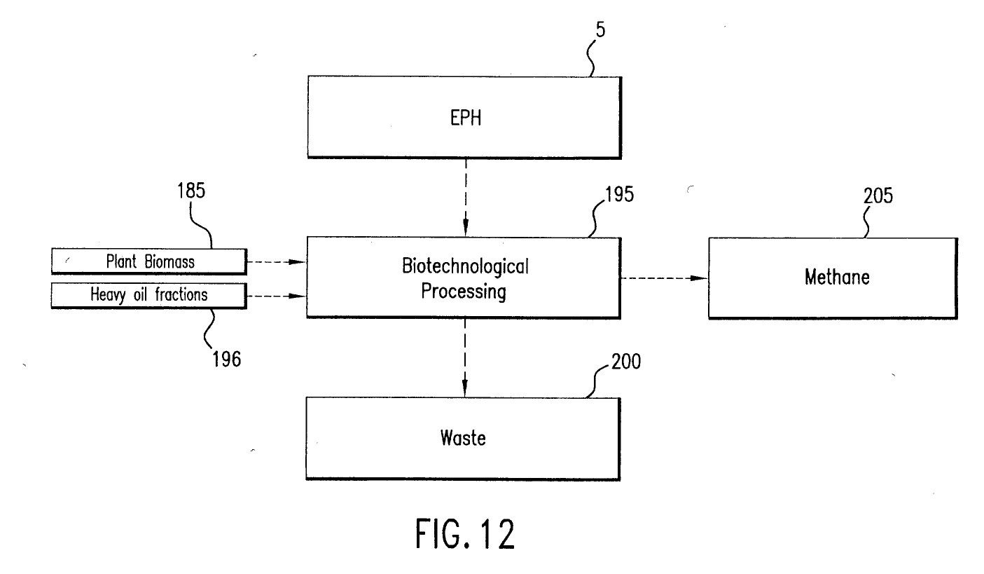

EPH is a nutrient medium and “growth” supplement for the intensive growth of microorganisms that produce methane and other natural gases. The gases produced by biological processes can be called collectively biogas. Referring to FIG. 6 and FIG. 12, addition of EPH into the reproduction phase, growth phase, and/or decline phase of biological organisms that produce biogas will enhance the speed and efficiency of the production process.

The production of methane (biogas) by the method of anaerobic processing of vegetable raw material and products of vital activity of humans and animals by digestion with the use of various bacteria and yeasts is the main example of the biological technology for the processing of wastes into “premium fuels.” If it is assumed that the initial raw material is glucose, the general scheme for the transformations to be accomplished can be represented as follows:

The production of biogas: C6H12O6[probably: —>] 3(CH4)+3(CO2); i.e., three molecules of methane (biogas) are obtained from a single glucose molecule. The heat of combustion of glucose is 16 kJ/g, while that of methane is 56 kJ/g, which provides grounds for the statement that the specific energy content in biogas is 3.5 times higher than in the initial substrate-the waste biomass.

In anaerobic reactors, it is possible to process an extremely wide variety of raw material which either is very inexpensive or has no commercial value at all: wood pulp waste, straw, rinds, domestic refuse, edible garbage, manure, sewage, brown algae, rice hulls, sugar cane bagasse, sugar cane, corn husks, corn kernels, wheat, canola, and many other grains, grasses, and plant materials. On an international scale, processing of this kind makes the greatest contribution to the energy budget of the country in China, where more than seven million small (single-family) reactors have been built.

Unpurified biogas is normally used as fuel in stationary plants for generating electric power. Compressed gas in tanks is suitable for fuel in all internal combustion engines.

Plants for the production of biogas can be grouped as follows according to increasing capacity:

1. reactors in rural areas in developing countries (which normally have a capacity of 1-20 m3);

2. reactors on farms in developed countries (capacity of 50-500 m3);

3. reactors for processing industrial wastes (capacity of 500-10,000 m3).

A large number of different kinds of plants are currently in use in developed countries. They include both small reactors and large-scale plants with equipment for gas purification, electric generators and compressors. The main task of all the designs is to maintain a high concentration of viable bacteria and not to allow losses of microorganisms in the operation of the system. The conversion of raw material to methane occurs in the course of complex interactions in mixed populations of microorganisms, where the main role, naturally, is played by methanogenic bacteria.

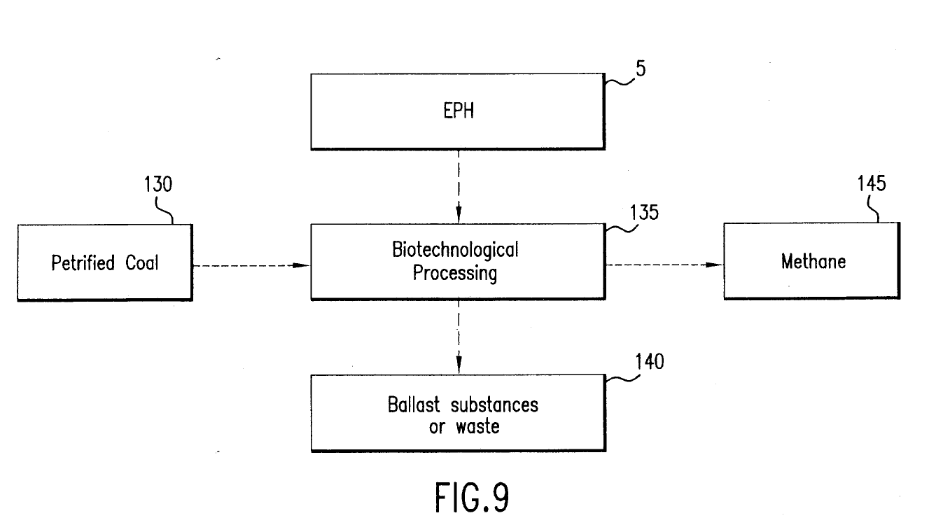

Referring to FIG. 9, EPH added to the reproductive phase, growth phase and decline phase of bacteria used to treat plant waste materials will lead to accelerated methane production. In the formation of methane, where the substrate is glucose, the gas yield by weight is approximately 27%, while the energy yield (theoretically) is more than 90%. In practice, however, due to the complex composition of the raw material which is processed in anaerobic reactors and the low efficiency of the conversion process, the gross energy yield is from 20 to 50%. In recent reports on plants for the conversion of wastes of varying quality into biogas, yields of 0.17 to 0.4 m3 of methane per kilogram of dry matter of the raw material are cited. The charging rate in the process was from 1 to more than 10 kg of raw material per cubic meter of the reactor per day, the retention time was 10-40 days, and the substrate conversion depth was from 20 to more than 70%.

No one anywhere in the world has been conducting intentional control of the composition of the substrate mixtures in reactors for producing biogas for the purpose of strengthening the growth of methane-producing bacteria. The quantity and viability of these bacteria depend directly on the concentrations of free amino acids in the substrates. Unfortunately, all the wastes used for processing are extremely poor in free amino acids; therefore, the methanogenic bacteria obtain the amino acids by way of a complex chain of metabolism of the other microorganisms.

Hence the biogas yield cannot be improved substantially even in the most modem plants without solving a basic problem of biotechnology, e.g., rapidly increasing the biomass of methane-producing bacteria. The use of EPH obtained by the enzymatic hydrolysis of raw material of fish and other animal origin is opening up enormous possibilities in this regard.

EPH may enhance the process to use certain bacteria for methane fermentation that has been described in U.S. Pat. No. 4,540,666, by Nukina, et al, which is hereby incorporated by reference in its entirety.

EPH may enhance the process to use certain bacteria for converting organic waste by thermophilic fermentation that has been described in U.S. Pat. No. 6,200,475, by Chen, which is hereby incorporated by reference in its entirety.

This EPH, which contains up to 75% free amino acids and bacterial growth factors, is a versatile nutrient medium suitable for all types of aerobic and anaerobic bacteria. The addition of EPH in balanced quantities along with the appropriate methanogenic or other biogas producing microbes to all types of wastes to be used for conversion to biogas will make it possible to make a sharp increase in the charging rate per cubic meter of the reactor per day; to achieve a substantial reduction in the retention time for raw material in the reactor; and to achieve a maximum increase in the substrate conversion depth, which, in the final analysis, will substantially increase the biogas yield and the gross production of energy by reactors of all types.

### EXAMPLE 9

### The Complex Technology For Enhancing the Production of Methane From Coal By the Addition of EPH With or Without Methanogenic Organisms

EPH is a nutrient medium and “growth” supplement for the intensive growth of microorganisms that produce methane and other natural gases. The addition of EPH into the reproduction phase, growth phase, and/or decline phase of biological organisms that produce methane from coal, including brown coals, lignites and related carbonaceous materials, in retorts or other vessels after coal is extracted from the ground will enhance the speed and efficiency of the methane production process.

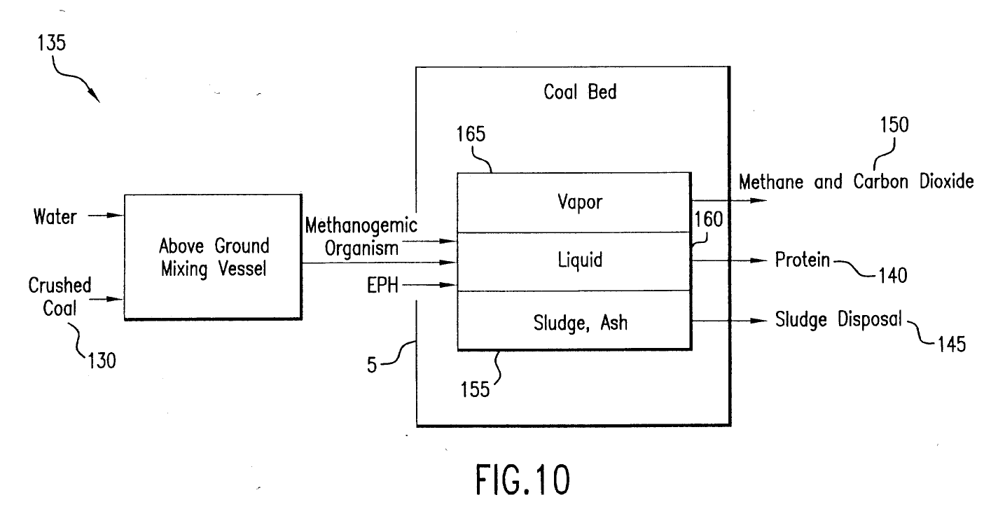

Referring to FIG. 10, injection of EPH and one or more types of methanogenic organisms, will provide free amino acids, along with a variety of minerals and vitamins, that will enable the organisms to reproduce, grow and proliferate without the limitations imposed by a severe lack of naturally occurring free amino acids in the coal. The need to add methanogenic organisms depends on how effective indigenous microflora are in bioassimilating coal. EPH will accelerate the rate of gas production due to the enhanced quantity of organisms and their relative strength. The concentration of EPH added to liquid 160 within coal bed 5 will be less than 4%, and typically below 1%. Methane and carbon dioxide 150 are collected from vapor 165 while sludge, ash 155 can be removed to maintain sufficient void space for the transport of gas to the surface.

EPH may enhance the process to use certain anerobic bacteria to gasify lignite that has been described in U.S. Pat. No. 6,143,534, by Menger, et al, which is hereby incorporated by reference in its entirety.

### EXAMPLE 10

### The Complex Technology For Enhancing the Production of Coal Bed Methane By the Injection of EPH With or Without Methanogenic Organisms

EPH is a nutrient medium and “growth” supplement for the intensive growth of microorganisms that produce methane and other natural gases. As shown in FIG. 10, addition of EPH into the reproduction phase, growth phase, and/or decline phase of biological organisms that produce methane from coal will enhance the speed and efficiency of the production process.

The method for injection is to include EPH within the materials used to fracture a coal seam in order to extract methane. The EPH, with or without the addition of one or more types of methanogenic organisms will provide free amino acids, along with a variety of minerals and vitamins, that will enable the organisms to reproduce, grow and proliferate without the limitations imposed by a severe lack of naturally occurring free amino acids in the coal bed. EPH will accelerate the rate of gas production due to the enhanced quantity of organisms and their relative strength. The concentration of EPH within the solution injected into the coal bed well will be less than 4%, and typically below 1%.

EPH may enhance the microbial process for producing methane from coal that has been described in U.S. Pat. No. 6,143,534, by Menger, et al, which is hereby incorporated by reference in its entirety.

### EXAMPLE 11

### The Complex Technology For Enhancing the Production of Ethanol From Plant Biomass By the Addition of EPH

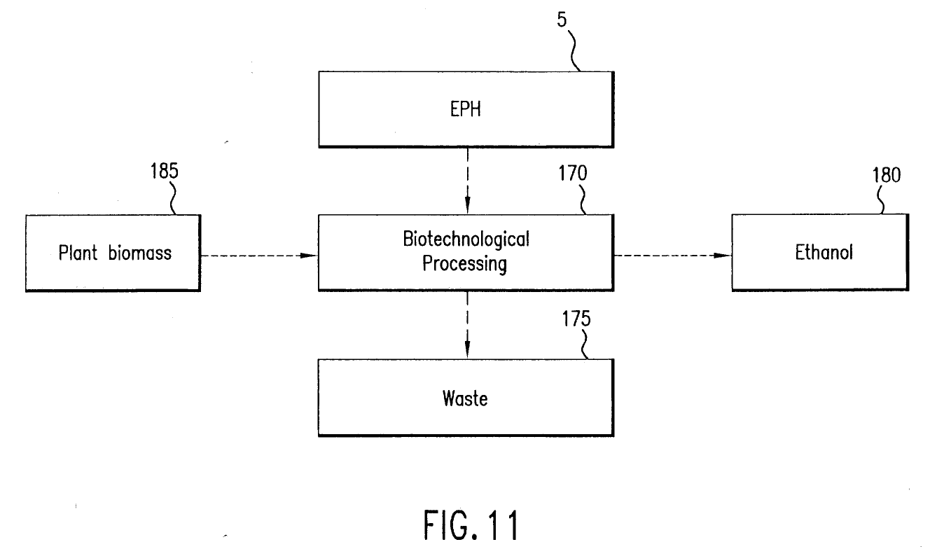

Microbial processes are frequently used to produce ethanol from sugars by fermentation. Several steps in the manufacture of finished products from sugar cane and similar high sugar content plant materials can be improved by the addition of EPH to the processes, as shown in FIG. 11. One such process improves the yield and significantly reduces the processing time for the fermentation of sugar to ethanol.

The addition of EPH into the reproduction phase, growth phase, and/or decline phase of biological organisms will enhance the speed and efficiency of the fermentation process, resulting in higher volumes of ethanol and less waste material. If used in conjunction with biological agents that remove lignin from plant materials, there should be a higher volume of sugar for fermentation than is typically available through conventional methods. Bagasse that results from the removal of sugar from cane can be treated with lignin removing organisms that are enhanced with EPH for optimal operation. With lignin removed, the remaining materials are primarily residual sugars and cellulose. The cellulose can be used for paper manufacturing and the sugars can be fermented into additional ethanol. The remaining residual vegetable matter can be composted and used as a high quality fertilizer containing significant amounts of EPH that are excellent nutrition components for plant life.

EPH may enhance the process for producing ethanol from recombinant hosts that has been described in U.S. Pat. No. 5,554,520, by Fowler, et al, which is hereby incorporated by reference in its entirety.

EPH may enhance the process for producing ethanol using soy proteins that has been described in U.S. Pat. No. 6,130,076, by Ingram, which is hereby incorporated by reference in its entirety.

### EXAMPLE 12

### The Complex Technology For Purifying Coal By the Removal of Nitrogen, Sulfur and Toxic Metals From Coal Via Biological Organisms in an EPH-enhanced Solution

EPH is a nutrient medium and “growth” supplement for the intensive growth of microorganisms that ingest sulfur compounds, nitrogen compounds and toxic metals present in coals, lignites, brown coals and related materials typically burned to produce energy. The addition of EPH into the reproduction phase, growth phase, and/or decline phase of such biological organisms will enhance the speed and efficiency of the coal purification process, resulting in cleaner burning materials with lower pollution potential than coals that are not treated.

The method for pretreatment is to include EPH with appropriate organisms for the coal material to be treated. Such materials vary widely in the sulfur, nitrogen and other toxic materials, so the appropriate organisms will vary accordingly. What unites the process is that fact to EPH will enhance the activity of all such organisms. The EPH, with or without the addition of one or more types of organisms will provide free amino acids, along with a variety of minerals and vitamins, that will enable the organisms to reproduce, grow and proliferate without the limitations imposed by a severe lack of naturally occurring free amino acids in coal. EPH will accelerate the rate of gas production due to the enhanced quantity of organisms and their relative strength.

The concentration of EPH within the solution injected into the coal bed well will be less than 4%, and typically below 1%. An illustrative method would be to crush 1,000 tons of coal into powder form prior to burning. A spray mechanism would be used to apply EPH-enhanced organisms on the coal evenly. After an appropriate period of time to permit the interaction of the organisms with the coal, the powdered coal would be rinsed with clear water to remove the EPH, organisms and polluting compounds. The remaining materials should be hydrocarbon coals with reduced quantities of sulfur, nitrogen and toxic metals.

EPH may enhance the process for microbial desulfurization of coal that has been described in U.S. Pat. No. 4,206,288, by Detz and U.S. Pat. No. 5,094,668, by Kern, which are hereby incorporated by reference in their entirety. In addition, EPH may enhance the process for biochemical transformation of coal that has been described in U.S. Pat. No. 5,885,825, by Liu, which is hereby incorporated by reference in its entirety.

### EXAMPLE 13

### EPH Enhancement of Biomass Conversion to Various Fuels, Including Methanol, Ethanol, Methane, Hydrogen and Related End Products

Another complex technology is the production of energy carriers. Input material for energy carriers may consist of animal, plant, fungal, or bacterial biomass, common or industrial waste, agricultural waste (rotten plant or food products), industrial sewage containing sugars or liquid waste created in sugar production or that of palm oil, waste from cities or alcohol factories, combustible slate, oil or its fractions, or other chemical substances and useful fossil fuels. The addition of EPH into the reproduction phase, growth phase, and/or decline phase of biological organisms that act on energy carriers will enhance the speed and efficiency of the energy conversion process, resulting in cleaner burning materials with lower pollution potential than processes without EPH.

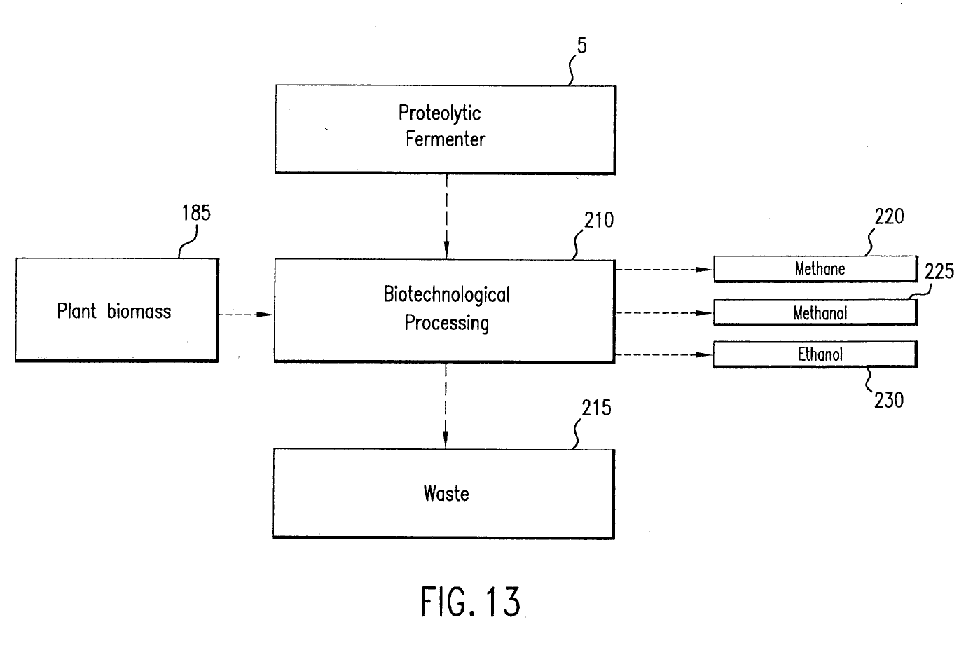

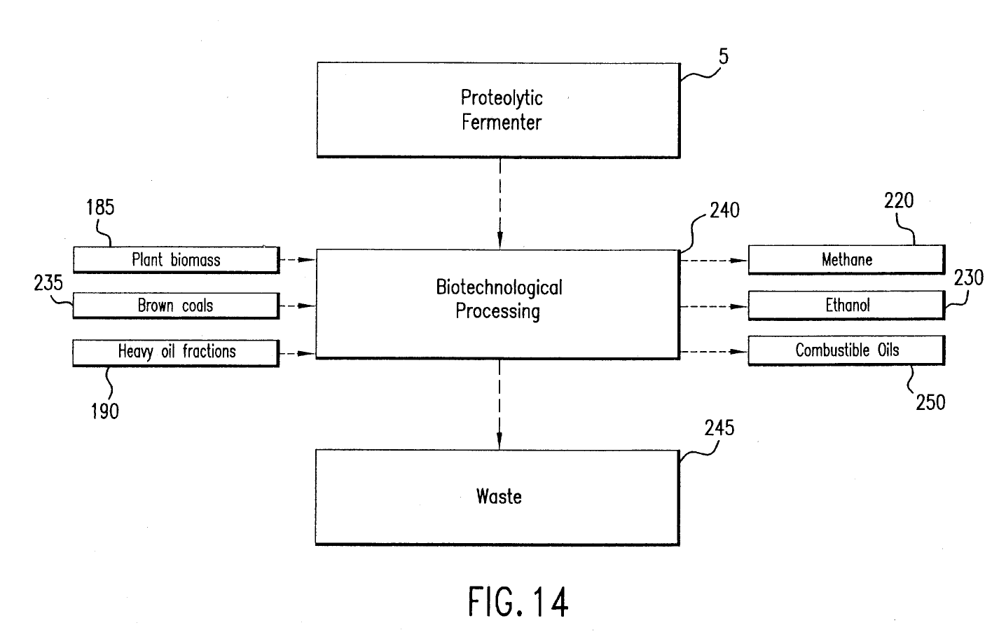

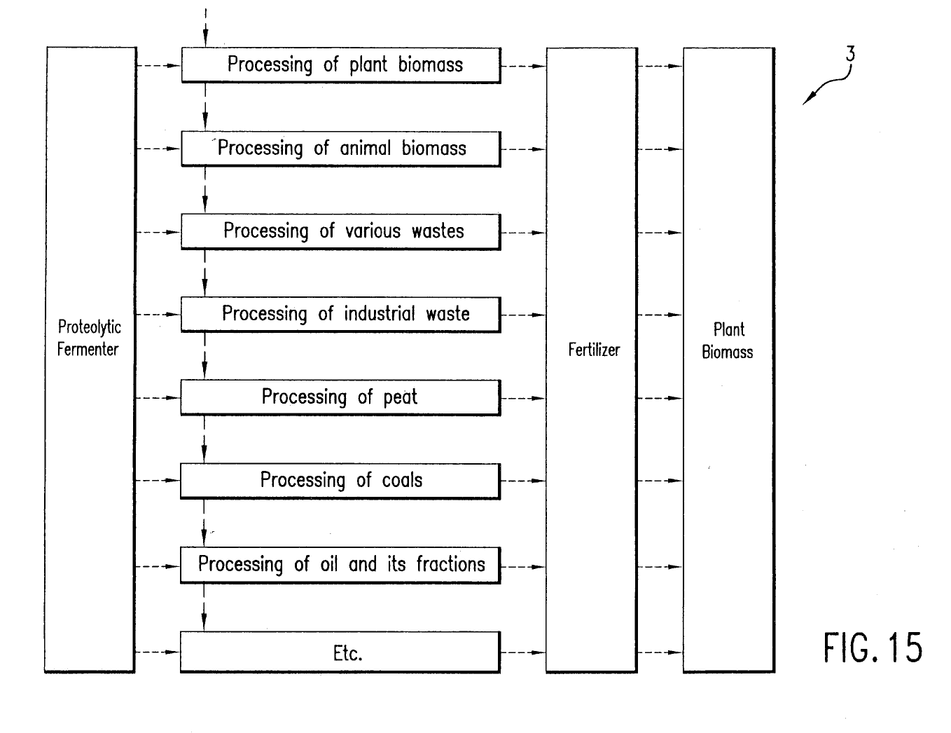

As shown in FIG. 13, FIG. 14, and FIG. 15, energy carriers also vary in type—methane, ethane, propane, butane, or other gases of this group, products of various bacterial activities, palm oil, methyl or ethyl alcohols, latexes, hydrogen, ammonia, methanol, ethanol, propanol, buthanol, various oils, long molecular alcohols, products like gasoline, kerosene or diesel fuel, etc.

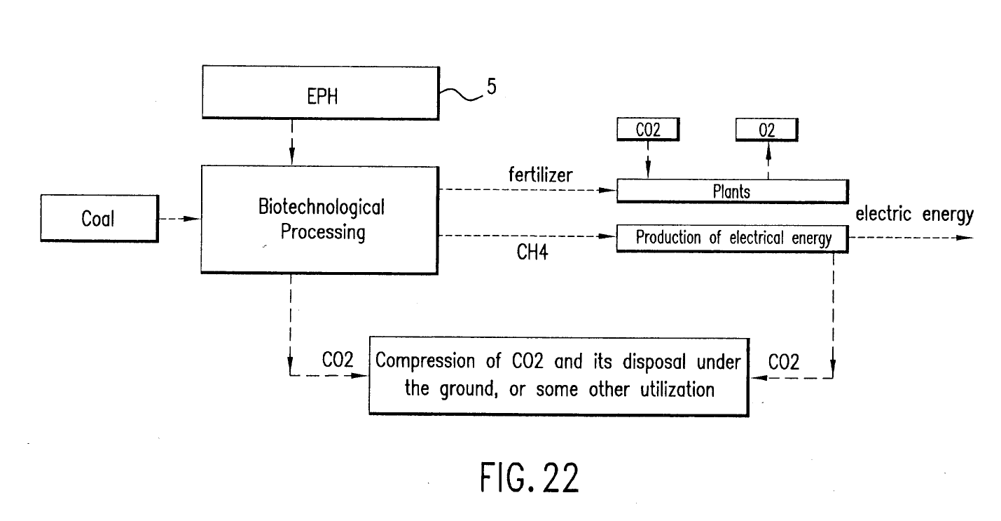

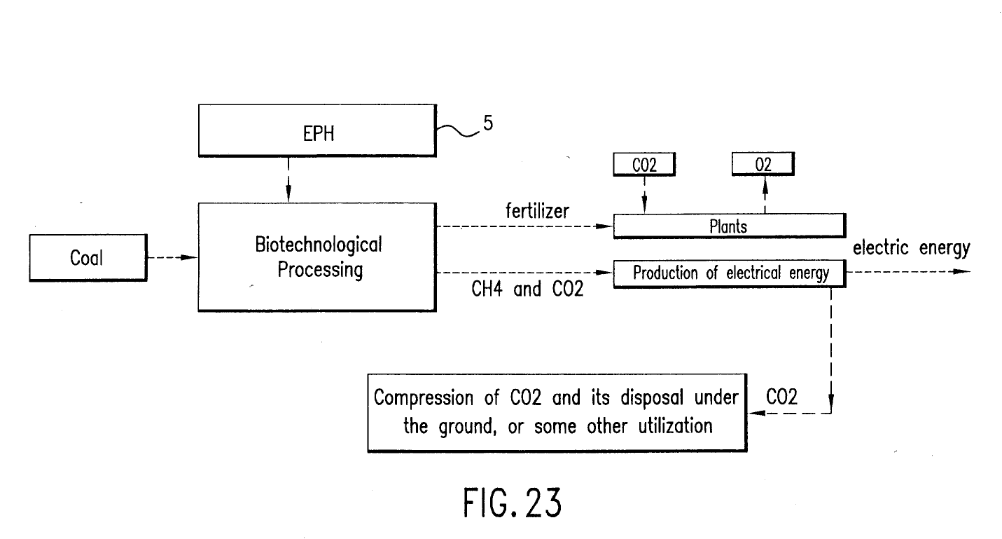

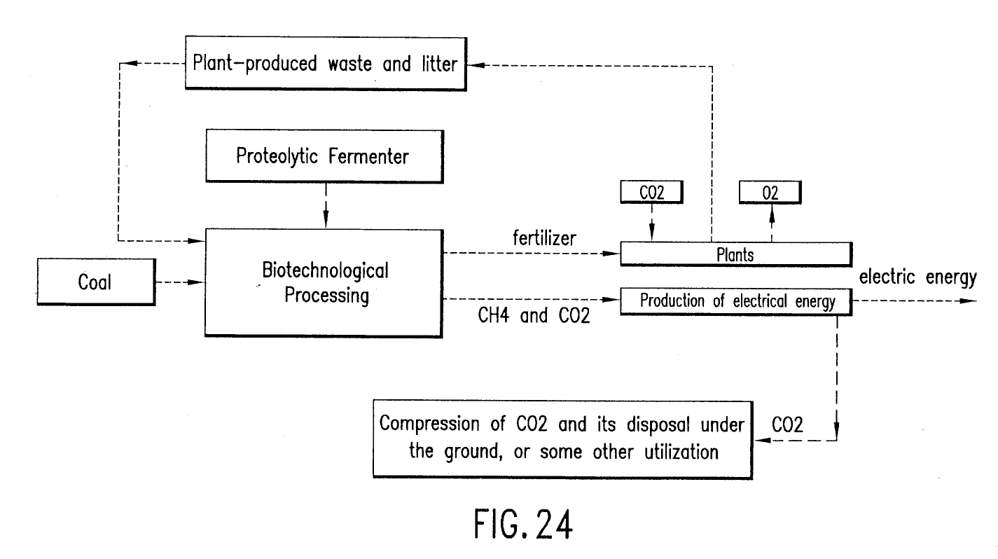

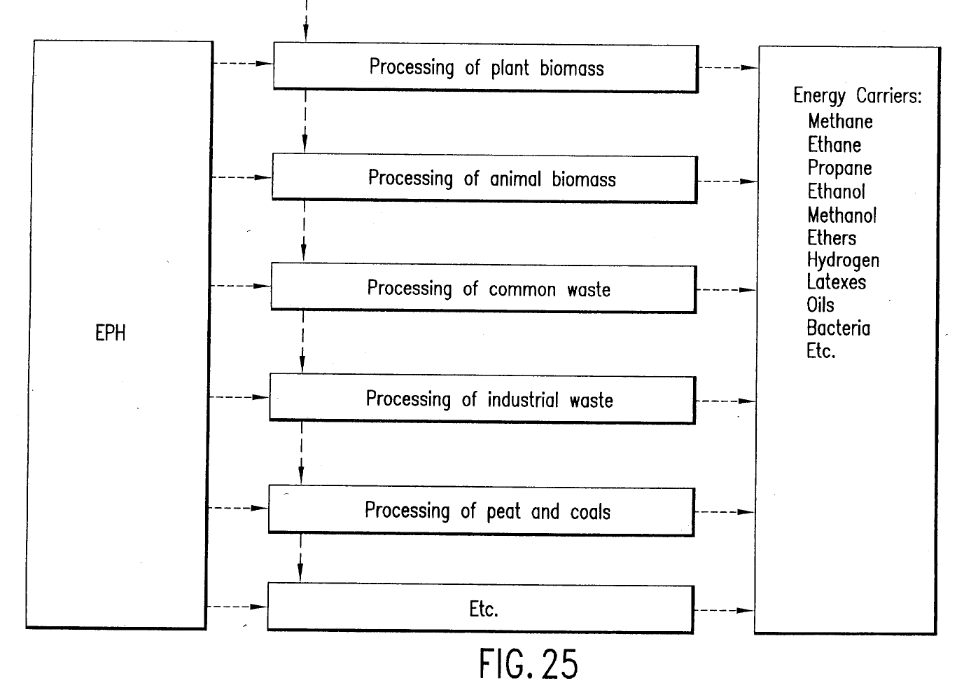

The complex technology of production of energy carriers may be used as a whole unit, or just one part of it. One example is the biotechnological processing of coal into methane, using EPH. Another example is the biotechnological processing of plant biomass into ethanol, using EPH. Otherwise, it can be a combination of several input materials to obtain a certain energy carrier; for instance, the biotechnological processing of heavy oil fractions and plant biomass into methane. Another option is derivation of several energy carriers from a single type of input material, for example biotechnological processing of plant biomass into methane, methanol, hydrogen and ethanol. As shown in FIG. 22, FIG. 23, FIG. 24, and FIG. 25, there can be derivation of several energy carriers from several types of input materials. For instance, biotechnological processing of heavy oil fractions, brown coal, and plant biomass into methane, ethanol, and combustible oils.

The complex technology for production of energy carriers is considered such not only because it combines several homogeneous technologies—other reasons also exist. This complex technology provides energy carriers accompanied by the processing of waste with a lower level of environmental pollution by chemical substances (serum oxide, ozone, nitrogen oxides, various heavy particles—Al, Fe, Ca, Si, C, Sb, Se, Zb, Pb, etc. carbon monoxide, polynuclear aromatic carbon hydroxides, etc.). In addition, there is the production of various input supplies for manufacturing of materials and chemical substances, the production of fertilizer, and a lower level of CO₂ in the atmosphere which may reduce the greenhouse effect and act as a means of carbon sequestration.

EPH may enhance the process for microbial solubilization of coal that has been described in U.S. Pat. No. 4,914,024, by Strandberg and U.S. Pat. No. 4,882,274, by Pyne, et al, which are hereby incorporated by reference in their entirety. EPH may enhance the process for the production of hydrogen by microorganisms that has been described in U.S. Pat. No. 5,464,539, by Ueno, et al, which is hereby incorporated by reference in its entirety

### EXAMPLE 14

### Complex Technology For the Cleansing of the Environment From Pollution and Waste Destruction, Growth of Plant (and Other) Biomass and Lowering the Atmospheric Level of CO2 and Production of Ecologically Uncontaminated Energy Carriers and Energy Through the Addition of EPH

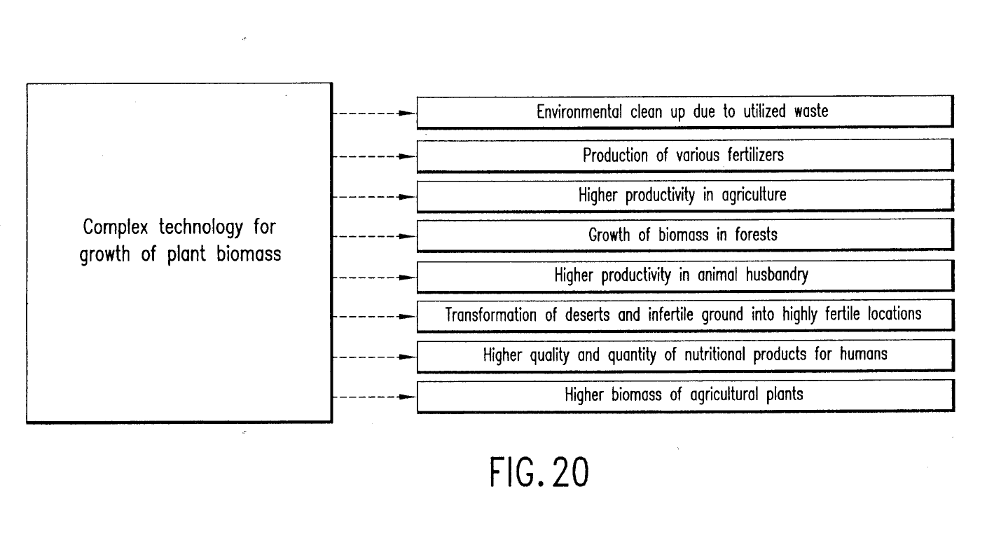

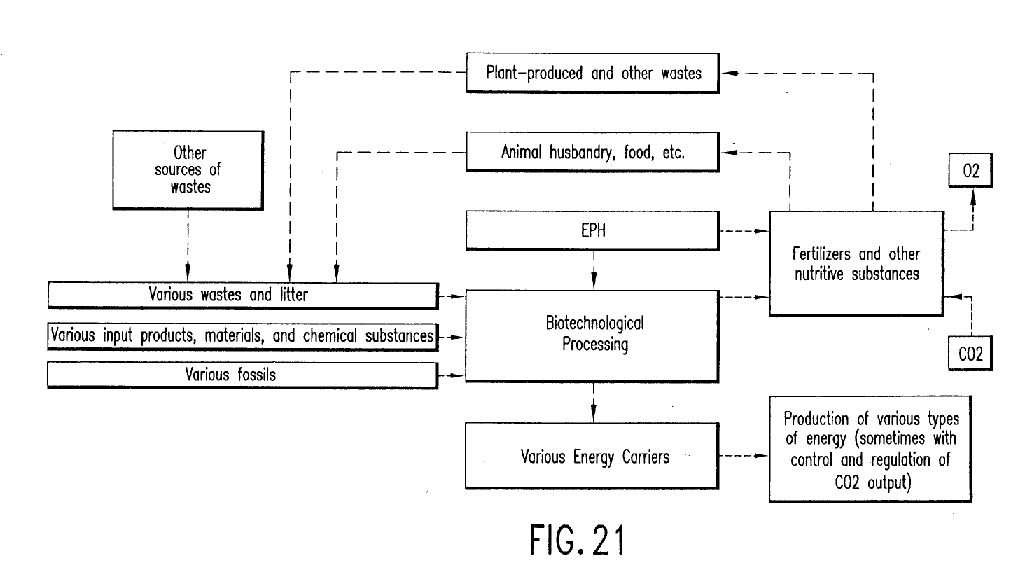

This complex technology is a synthesis or several biological processes, all of which are enhanced by the addition of EPH. The addition of EPH into the reproduction phase, growth phase, and/or decline phase of biological organisms unites the three complex technologies described above. This technology can be realized as either a whole unit or one of its parts, as shown in FIG. 20 and FIG. 21. In other words, it can exist as any combination of input ingredients (fossil fuels, wastes, etc.) as any combination of steps, cycles and devices of intermediary processing, as any combination of end ingredients, products, and devices. Also, any parts of this complex technology can be connected to any simple or complex technology, the technologies can be united with other technologies into various technological chains, etc.

### EXAMPLE 15

### Complex Technology For Use of EPH in Biotechnology

The culturing of microorganisms and cells of various human and animal tissues is the basis for one of the most timely fields of human knowledge, biotechnology. The culturing process is conducted in special containers in bioreactors filled with culture media that contain the entire complex of substances required for the normal functioning and reproduction of the microorganisms and cells. All the culture media are “disposable,” i.e., are used only once; hence the level of consumption of such media in biotechnology, especially on a commercial scale, is extremely high and runs into the hundreds of tons. The basis for practically all culture media is EPH. The addition of EPH into the reproduction phase, growth phase, and/or decline phase of biological organisms used in biotechnology will ensure that organisms have the mixture of free amino acids, vitamins, minerals to ensure their optimal growth, regulation, and reproduction.

The requirements for nutrient media, which value not only the availability of nutrients but also the presence of specific growth factors, without which the productivity of the cells is sharply reduced, are especially high in a comparatively new field of biotechnology: the culturing of human and animal cells. EPH fully satisfies these high requirements. Nutrient media based on EPH supports the growth and development of up to 40 generations of animal and human cells.

Nutrient media for culturing microorganisms supports the growth of the biotechnology industry. Without this field of biotechnology, it would be impossible to produce vaccines, genetic engineering products or bacterial preparations. This field of biotechnology is the largest consumer of nutrient media. The public health service of any country also has an urgent need for nutrient media of this kind, since the diagnosis of infectious diseases is impossible without them. The addition of EPH into the reproduction phase, growth phase, and/or decline phase of biological organisms within nutrient media is entirely suitable for the culturing of all known bacterial and protozoan infectious agents.

EPH may enhance the process for animal cell culture that has been described in U.S. Pat. No. 5,672,502, by Birch, et al, which is hereby incorporated by reference in its entirety.

### EXAMPLE 16

### Complex Techology for Animal Breeding And Veterinary Medicine

The addition of EPH into the reproduction phase, growth phase, and/or decline phase of biological organisms can be especially efficacious in animal breeding and veterinary medicine. For all farm animals, and especially for young animals, EPH can serve as a basic source of the essential amino acids, water-soluble vitamins, minerals and trace elements which determine not only the normal growth, development and reproductive capacity of the animals, but also the quality of the products obtained from the animals (milk, meta, hides, wool and fur).

For domestic carnivores (dogs and cats), EPH is a vitally necessary food supplement, since the animals may suffer chronic vitamin and mineral deficiencies when they are fed canned or preserved foods. Such a diet has an especially harmful effect on the health of young, growing animals; it retards the growth of their external features, sharply damages the quality of their coats, depresses their emotions and generally shortens their lives.

In the breeding of fur-bearing animals (the breeding and raising of mink, fox, weasel, Arctic fox, sable and nutria in cages), EPH, with its ideal set of essential amino acids and B vitamins, is an effective means for improving the quality of the skins by improving the thickness, lushness and strength of the fur.

In commercial poultry farming, essential amino acids, B vitamins and minerals have an extremely important role in a balanced diet for all types of birds, since they determine the health of the fast-growing young and, most important, the strength of the shell of the egg.

In commercial fish breeding, EPH can serve as the basis for the development of a fundamentally new feed for the young fish (especially for species of salmon), as well as for adult fish. The rapid assimilation of the EPH in the body of the fish makes it possible to reduce substantially the time for raising commercial fish and to improve the quality of the fish.

It should be noted in particular that EPH can be used successfully in veterinary pharmacology in the development of new types of drugs and preventive and cosmetic preparations for animals.

### EXAMPLE 17

### Complex Technology of Obtaining Fossil Fuels

There are various ways of utilizing EPH to obtain fossil fuels. Often, specific substances are inserted into petroleum that change its rheological characteristics, or some other features that increase the effectiveness of acquiring this fossil fuel. These substances can be obtained as products of bacterial activity. The addition of EPH into the reproduction phase, growth phase, and/or decline phase of biological organisms can accelerate this bacterial activity. In this case, as in many others, EPH can be used for the preparation of products, substances, and materials that improve the quality of traditional methods of obtaining fossil fuels.

In other cases, the proteolytic machine can be used for the production of an absorbent substance based on humic acid to obtain certain fossil fuels contained in a body of water (sea, ocean, underground, river).

With the same goal in mind, EPH can be used to obtain chitosan from the exoskeletons of marine crabs. In this and many other cases EPH can be used for the preparation of products, substances, and materials that facilitate acquisition of fossil fuels by non-traditional methods.

In other cases, EPH can be used to increase the rate of reproduction and growth of those organisms that can independently uncover fossil fuels from subterranean locations, ocean waters, or other sites. Thus, the use of bacteria allows us to obtain many metals—gold, molybdenum, wolfram, silver, uranium, etc., as well as various non-metals. The methods of biotechnological acquisition of fossils can be adequately used in developed mines where it is impossible to unearth the desired fossils by traditional methods. In these cases, EPH is used for direct acquisition of fossils through biotechnological methods.

### EXAMPLE 18

### Complex Technologies of Production of Various Mixtures, Products, Chemical Substances, and Materials

The addition of EPH into the reproduction phase, growth phase, and/or decline phase of biological organisms can stimulate bacterial activity that produces various chemical substances. Moreover, certain chemical substances may be derived from plants, fungi, and animal organs. Again, EPH can be used to accelerate their activity.

In this case, EPH is used for activation of vitality of various substances' producents. EPH can either be used directly—to add amino acids into the producents' environment, or indirectly—by activating with amino acids the supply of some other supplement for these producents and providing them with some other improvements in their surroundings, excluding nutrition.

In other cases, amino acids may be added to existing mixtures and combined with a given substance. For instance, they may be added to packaging material to make that item more biodegradable. They can also be combined with cellulose of with humic acid to obtain effective fertilizers. In this case, EPH can be used for creating mixtures of substances and amino acids.

Finally, amino acids can be combined with bacterial mass such as a mixture of Devoroils and amino acids. Devoroil is a mixture of bacteria and is used for soil decontamination of oil products. Addition of amino acids increases the effectiveness of this product, also changing its basic nature. Amino acids can be used in combination with seaweeds in decontamination mechanisms in the same manner. Amino acids can also be combined with yeast to increase the effectiveness of the latter, etc. In this case, EPH can be used for the creation of bioproducts. This category includes various pharmaceutical products: bifidus bacterium, coly-bacterium, etc.

### EXAMPLE 19

### Landfill Methane

As outlined in some of the sections above, the addition of EPH into the reproduction phase, growth phase, and/or decline phase of biological organisms that produce methane can improve the production of methane from landfills. Landfill methane is a clean fuel and the government is committed to expanding its availability. EPH can be injected into existing landfill methane operations to accelerate the rate of production.

### EXAMPLE 20

### Rendering Process Improvements

EPH can be utilized in digestive and fermetation reactions to improve the yield of natural decomposition processes. Rendering processes can be improved by the addition of EPH into the reproduction phase, growth phase, and/or decline phase of biological organisms that convert animal wastes into compost, oils, fats, and additional amino acid containing compounds. Potential raw materials come from pigs, cows, sheep, turkeys, shrimp and many other commercial animals.

### EXAMPLE 21

### Animal Wastes

The use of EPH in combination with bacteria that provide odor control and degradation of raw manure has immediate applications in the poultry production industry. The direct application of such a mixture can be used immediately and in large quantities at chicken farms. In addition, EPH can be used in combination with bacteria that generate methane to treat pig manure. Ultimately, EPH can serve as the catalyst for the development of a comprehensive program for improving farming and animal husbandry methods in general.

### EXAMPLE 22

### Septic/Wastewater Systems

The biological processes used to treat septic wastes in local and municipal wastewaters as well as wastewater streams derived from a number of municipal and industrial wastes can also be enhanced by the use of EPH. The addition of EPH into the reproduction phase, growth phase, and/or decline phase of biological organisms that act in such treatment systems will accelerate treatment time and permit higher volumes of materials to be biologically treated that previously.

### EXAMPLE 23

### Remediation of Hazardous Wastes

Numerous processes have been invented in recent years to utilize biological organisms to to clean up hazardous waste sites or oil spills. The addition of EPH into the reproduction phase, growth phase, and/or decline phase of such biological organisms can accelerate their activity and promote more rapid and effective hazardous waste remediation.

### EXAMPLE 24

### The Complex Technology For Enhancing the Production Removal of Lignin From Pulp to Produce Pure Cellulose By the Addition of EPH

The addition of EPH into the reproduction phase, growth phase, and/or decline phase of biological organisms will enhance the speed and efficiency of the biological agents that remove lignin from plant materials. Pulp typically contains a significant amount of lignin, which must be removed by chemical or biological means in order to isolate cellulose for paper production. With lignin removed by microbial agents whose enzyme activity is enhanced by the addition of EPH, the remaining materials are primarily residual sugars and cellulose. The cellulose can be used for paper manufacturing and the sugars can be fermented into ethanol, if desired. The remaining residual vegetable matter can be composted and used as a high quality fertilizer containing significant amounts of EPH that are excellent nutrition components for plant life.

We claim:

1. A method for accelerating the growth of microorganisms employed in industrial processes, comprising:
adding an enzymatic protein hydrolysate (EPH) to said microorganisms during a growth phase of said microorganisms.

2. The method according to claim 1, wherein said EPH comprises a 70-90 percent mixture, by weight, of free amino acids, a 10-20 percent mix, by weight, of bioactive peptides, and a 3-5 percent mixture, by weight, of vitamins and minerals.

3. The method of claim 2, wherein said amino acids consist of: taurine, aspartine, threonine, serine, glutamic acid, praline, glycine, alanine, valine, methionine, isoleucine, leucine, tyrosine, phenylaline, oxyhedral proline, omithine, lysine, histidine, arginine.

4. The method of claim 3, wherein said minerals consist of: cadmium, lead, mercury, arsenic, natrium, potassium, calcium, phosphorous, magnesium, iron, zinc, copper, manganese, selenium, cobalt, molybdenum, and chromium.

5. The method according to claim 1, wherein said microorganisms are bacteria.

6. The method according to claim 2, wherein said microorganisms are bacteria.

7. The method according to claim 3, wherein said microorganisms are bacteria.

8. The method according to claim 4, wherein said microorganisms are bacteria.

9. The method according to claim 1, wherein said microorganisms are yeasts.

10. The method according to claim 2, wherein said microorganisms are yeasts.

11. The method according to claim 3, wherein said microorganisms are yeasts.

12. The method according to claim 4, wherein said microorganisms are yeasts.

13. The method according to claim 1, wherein said microorganisms are fungi.

14. The method according to claim 4, wherein said microorganisms are fungi.

15. The method according to claim 4, wherein said EPH is an additive to the final product of said industrial processes, wherein said product is selected from among the group consisting of: food colorants, food flavorings, vitamins, vegetable-derived pastes, food thickeners, meat products, fish products, fruits, vegetable preserves, pet foods, food spices, vinegar, lactic acid, salicylic acid, amber, apple, enzymes, chemical substances, and biopolymers.

16. The method according to claim 8, wherein said EPH is added as an accelerant to said bacteria that are utilized in the processing of industrial waste, wherein said waste is selected from the group comprising: plant biomass, animal biomass, industrial waste, sewage, xenobiotics, and crude oil.

17. The method according to claim 8, wherein said EPH is added as an accelerant to said bacteria that are utilized in the processing of metals—bioextractive metallurgy—the processing of metals selected from the group including: uranium, gold, silver, copper, zinc, cobalt, and rare earth metals.

18. The method according to claim 8, wherein said EPH is added as an accelerant to said bacteria utilized in the production of ethyl alcohol, methyl alcohol, prophyl alcohol, methane gas, acetone, butanol, isopropyl, diesel fuel.

19. A method for enhancing the production of coal, comprising:
adding EPH to any mixture of coal digesting microorganisms during their growth phase.

20. The method according to claim 19, wherein said microorganisms are derived from the digestive tract of termites.
